SEQUENCE LISTING (28 sequences)

SEQ ID NO: 1 - DNA, 346 bp, Artificial Sequence
  Synthetic
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccgggga

SEQ ID NO: 2 - DNA, 423 bp, Artificial Sequence
  Synthetic
gtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctatcgatcggccggataacgccacc

SEQ ID NO: 3 - DNA, 496 bp, Artificial Sequence
  Synthetic
gaattctccaggcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctccgcggggatcc

SEQ ID NO: 4 - DNA, 12 bp, Artificial Sequence
  Synthetic
attggatccaat

SEQ ID NO: 5 - DNA, 28 bp, Artificial Sequence
  Synthetic
tatttacaaacaaccatgaatgtaagta

SEQ ID NO: 6 - DNA, 18 bp, Artificial Sequence
  Synthetic
cggagtactgtcctccga

SEQ ID NO: 7 - DNA, 30 bp, Artificial Sequence
  Synthetic
atgatacgaaacgtaccgtatcgttaaggt

SEQ ID NO: 8 - DNA, 26 bp, Artificial Sequence
  Synthetic
agaaacaaaccaacctgtctgtatta

SEQ ID NO: 9 - DNA, 28 bp, Artificial Sequence
  Synthetic
aacaaacagacaatctggtctgtttgta

SEQ ID NO: 10 - protein, 249 aa, Artificial Sequence
  Synthetic
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYELTLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 11 - protein, 248 aa, Artificial Sequence
  Synthetic
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 12 - protein, 298 aa, Artificial Sequence
  Synthetic
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADSGGGGPTDALDDFDLDMLPADALDDFDLDMLPADALDDFDLDMLPGPPKKKRKV

SEQ ID NO: 13 - protein, 264 aa, Artificial Sequence
  Synthetic
MVRRTKEEAQETRAQIIEAAERAFYKRGVARTTLADIAELAGVTRGAIYWHFNNKAELVQALLDSLHETHDHLARASESEDEVDPLGCMRKLLLQVFNELVLDARTRRINEILHHKCEFTDDMCEIRQQHQSAVLDCHKGITLTLANVVRRGQLPGELDAERAAVAMFAYVDGLIRRWLLLPDSVDLLGDVEKWVDTGLDMLRLSPALRKSGGGGPTDALDDFDLDMLPADALDDFDLDMLPADALDDFDLDMLPGPPKKKRKV

SEQ ID NO: 14 - protein, 254 aa, Artificial Sequence
  Synthetic
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCSGGGGPTDALDDFDLDMLPADALDDFDLDMLPADALDDFDLDMLPGPPKKKRKV

SEQ ID NO: 15 - protein, 257 aa, Artificial Sequence
  Synthetic
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 16 - protein, 260 aa, Artificial Sequence
  Synthetic
MVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPVLREGIQRTVERNRFVVGDIWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 17 - protein, 432 aa, Unknown
  Lentivirus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQAN

SEQ ID NO: 18 - protein, 1003 aa, Unknown
  Lentivirus
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 19 - protein, 511 aa, Unknown
  Lentivirus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 20 - protein, 116 aa, Unknown
  Lentivirus
MAGRSGDSDEDLLKAVRLIKFLYQSNPPPNPEGTRQARRNRRRRWRERQRQIHSISERILSTYLGRSAEPVPLQLPPLERLTLDCNEDCGTSGTQGVGSPQILVESPTILESGAKE

SEQ ID NO: 21 - DNA, 578 bp, Artificial Sequence
  Synthetic
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 22 - DNA, 571 bp, Artificial Sequence
  Synthetic
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatc

SEQ ID NO: 23 - protein, 19 aa, Artificial Sequence
  Synthetic
ATNFSLLKQAGDVEENPGP

SEQ ID NO: 24 - protein, 18 aa, Artificial Sequence
  Synthetic
EGRGSLLTCGDVEENPGP

SEQ ID NO: 25 - protein, 20 aa, Artificial Sequence
  Synthetic
QCTNYALLKLAGDVESNPGP

SEQ ID NO: 26 - protein, 22 aa, Artificial Sequence
  Synthetic
VKQTLNFDLLKLAGDVESNPGP

SEQ ID NO: 27 - protein, 261 aa, Artificial Sequence
  Synthetic
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSPKKKRKVDYKDDDDKPADALDDFDLDMLPADALDDFDLDMLPADALDDFDLDML

SEQ ID NO: 28 - protein, 335 aa, Artificial Sequence
  Synthetic
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

(19) United States
(12) Patent Application Publication
(10) Pub. No.: US 2024/0191253 A1
(43) Pub. Date: Jun. 13, 2024

(54) STABLE PRODUCTION SYSTEMS FOR LENTIVIRAL VECTOR PRODUCTION

(71) Applicant: Asimov Inc., Boston, MA (US)

(72) Inventors: Jeremy J. Gam, Boston, MA (US); Alec A.K. Nielsen, Boston, MA (US)

(73) Assignee: Asimov Inc., Boston, MA (US)

(21) Appl. No.: 18/287,968

(22) PCT Filed: Apr. 22, 2022
(86) PCT No.: PCT/US2022/026004

§ 371 Date: Oct. 23, 2023

Related U.S. Application Data
(60) Provisional application No. 63/179,129, filed on Apr. 23, 2021.

(51) Int. Cl. C12N 15/86

(52) U.S. Cl. CPC C12N 15/86; C12N 2740/15043; C12N 2740/15051; C12N 2830/003

(57) ABSTRACT

Described herein are lentiviral vector production systems. Also described herein are engineered cells and kits comprising a lentiviral vector production system and methods of using the same for lentiviral vector production.

### FIELD

Described herein are lentiviral vector production systems. Also described herein are engineered cells and kits comprising a lentiviral vector production system and methods of using the same for lentiviral vector production.

### RELATED APPLICATIONS

This application claims the benefit under 35 U.S.C. § 119 of U.S. provisional application Ser. No. 63/179,129, filed Apr. 23, 2021, the entire contents of which are incorporated by reference herein.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Apr. 22, 2022 is named A121070003WO00-SEQ and is 45,885 bytes in size.

### BACKGROUND

Viral vectors are a promising gene delivery modality for cell and gene therapy. Viral vectors can be modified to carry therapeutic genetic payloads to cells within a subject. The production of viral vectors normally entails transient transfection of plasmids containing genes required for viral vector production into cell culture. However, transient transfection has several shortfalls. Large quantities of DNA and transfection reagent must be procured for the transfection process, which is costly. Also, poor transfection efficiency can result in minimal numbers of “transfected” cells and increased variation associated with transfection steps and viral production.

### SUMMARY

Described herein are lentiviral vector production systems. Also described herein are kits comprising a lentiviral vector production system. Finally, engineered cells comprising a lentiviral vector production system (or at least a portion thereof) are described, as well as methods of using the engineered cells for lentiviral vector production.

In some aspects, the disclosure relates to an engineered cell for lentiviral vector production comprising one or more stably integrated heterologous polynucleic acids collectively comprising: a nucleic acid sequence encoding for Gag; a nucleic acid sequence encoding for Pol; a nucleic acid sequence encoding for VSV-G; and a nucleic acid encoding for Rev; at least one of which is operably linked to a chemically inducible promoter. In some embodiments, the engineered cell comprising a chemically inducible promoter of the nucleic acid sequence of one or more of SEQ ID NOs: 1-9.

In some embodiments, the engineered cell comprises a nucleic acid sequence encoding for Gag operably linked to a chemically inducible promoter. In some embodiments, Gag comprises the amino acid sequence of SEQ ID NO: 17.

In some embodiments, the engineered cell comprises a nucleic acid sequence encoding for Pol operably linked to a chemically inducible promoter. In some embodiments, Pol comprises the amino acid sequence of SEQ ID NO: 18.

In some embodiments, the engineered cell comprises a nucleic acid sequence encoding for VSV-G operably linked to a chemically inducible promoter. In some embodiments, VSV-G comprises the amino acid sequence of SEQ ID NO: 19.

In some embodiments, the engineered cell comprises a nucleic acid sequence encoding for Rev operably linked to a chemically inducible promoter. In some embodiments, Rev comprises the amino acid sequence of SEQ ID NO: 20.

In some embodiments, the engineered cell comprises:
(a) a first stably integrated heterologous polynucleic acid comprising the nucleic acid sequence encoding for Gag and the nucleic acid sequence encoding for Pol;
(b) a second stably integrated heterologous polynucleic acid comprising the nucleic acid sequence encoding for VSV-G; and
(c) a third stably integrated heterologous polynucleic acid comprising the nucleic acid sequence encoding for Rev.

In some embodiments, the first stably integrated heterologous polynucleic acid comprises a first expression cassette comprising: (i) a nucleic acid sequence of a first chemically inducible promoter; and (ii) a nucleic acid sequence encoding for a polycistronic RNA, wherein the polycistronic RNA comprises the nucleic acid sequence encoding for Gag and the nucleic acid sequence encoding for Pol. In some embodiments, the first chemically inducible promoter comprises the nucleic acid sequence of one or more of SEQ ID NOs: 1-9. In some embodiments, the first stably integrated heterologous polynucleic acid further comprises a nucleic acid sequence of a selection marker that is operably linked to a nucleic acid sequence of a promoter.

In some embodiments, the second stably integrated heterologous polynucleic acid comprises a second expression cassette comprising: (i) a nucleic acid sequence of a second chemically inducible promoter; and (ii) the nucleic acid sequence encoding for VSV-G. In some embodiments, the second stably integrated heterologous polynucleic acid further comprises a nucleic acid sequence of a transcriptional activator operably linked to a nucleic acid sequence of a promoter, wherein the transcriptional activator, when expressed in the presence of a small molecule inducer, binds to the second chemically inducible promoter of the second expression cassette. In some embodiments, the second chemically inducible promoter comprises the nucleic acid sequence of one or more of SEQ ID NOs: 1-9. In some embodiments, the second stably integrated heterologous polynucleic acid further comprises a nucleic acid sequence of a selection marker that is operably linked to a nucleic acid sequence of a promoter.

In some embodiments, the third stably integrated heterologous polynucleic acid comprises a third expression cassette comprising: (i) a nucleic acid sequence of a third chemically inducible promoter; and (ii) the nucleic acid sequence encoding for Rev. In some embodiments, the third stably integrated heterologous polynucleic acid further comprises a nucleic acid sequence of a transcriptional activator operably linked to a nucleic acid sequence of a promoter, wherein the transcriptional activator, when expressed in the presence of a small molecule inducer, binds to the third chemically inducible promoter of the third expression cassette. In some embodiments, the third chemically inducible promoter comprises the nucleic acid sequence of one or more of SEQ ID NOs: 1-9. In some embodiments, the third stably integrated heterologous polynucleic acid further comprises a nucleic acid sequence of a selection marker that is operably linked to a nucleic acid sequence of a promoter.

In some embodiments, the engineered cell further comprises a heterologous polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the engineered cell. In some embodiments, the transcriptional activator comprises the amino acid sequence of any one of SEQ ID NOs: 10-16.

In some embodiments, the engineered cell comprises:
(a) a first stably integrated heterologous polynucleic acid comprising the nucleic acid sequence encoding for Gag and the nucleic acid sequence encoding for Pol; and
(b) a second stably integrated heterologous polynucleic acid comprising the nucleic acid sequence encoding for VSV-G and the nucleic acid sequence encoding for Rev.

In some embodiments, the first stably integrated heterologous polynucleic acid comprises a first expression cassette comprising: (i) a nucleic acid sequence of a first chemically inducible promoter; and (ii) a nucleic acid sequence encoding for a polycistronic RNA, wherein the polycistronic RNA comprises the nucleic acid sequence encoding for Gag and the nucleic acid sequence encoding for Pol. In some embodiments, the first chemically inducible promoter comprises the nucleic acid sequence of one or more of SEQ ID NOs: 1-9. In some embodiments, the first stably integrated heterologous polynucleic acid further comprises a nucleic acid sequence of a selection marker that is operably linked to a nucleic acid sequence of a promoter.

In some embodiments, the second stably integrated heterologous polynucleic acid comprises a second expression cassette comprising: (i) a nucleic acid sequence of a second chemically inducible promoter; and (ii) the nucleic acid sequence encoding for VSV-G; and a third expression cassette comprising: (i) a nucleic acid sequence of a third chemically inducible promoter; and (ii) the nucleic acid sequence encoding for Rev. In some embodiments, the second stably integrated heterologous polynucleic acid comprises: a second expression cassette comprising: (i) a nucleic acid sequence of a second chemically inducible promoter; and (ii) the nucleic acid sequence encoding for Rev; and a third expression cassette comprising: (i) a nucleic acid sequence of a third chemically inducible promoter; and (ii) the nucleic acid sequence encoding for VSV-G. In some embodiments, the second promoter comprises the nucleic acid sequence of one or more of SEQ ID NOs: 1-9; the third promoter comprises the nucleic acid sequence of one or more of SEQ ID NOs: 1-9; or a combination thereof. In some embodiments, the second stably integrated heterologous polynucleic acid further comprises a nucleic acid sequence of a selection marker that is operably linked to a nucleic acid sequence of a promoter.

In some embodiments, the engineered cell further comprising a heterologous polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the engineered cell. In some embodiments, the transcriptional activator comprises the amino acid sequence of any one of SEQ ID NOs: 10-16. In some embodiments, the second stably integrated heterologous polynucleic acid further comprises the nucleic acid sequence of transcriptional activator operably linked to a promoter.

In some embodiments, the engineered cell further comprises a stable landing pad. In some embodiments, the engineered cell is derived from a HEK293 cell, a HeLa cell, a BHK cell, or a Sf9 cell.

In some aspects, this application discloses a kit comprising an engineered cell as described herein.

In some embodiments, the kit further comprises a transfer polynucleic acid molecule comprising, from 5′ to 3′: (i) a nucleic acid sequence of a 5′ lentivirus long tandem repeat (LTR); (ii) a multiple cloning site; and (iii) a nucleic acid sequence of a 3′ lentivirus long tandem repeat (LTR). In some embodiments, the transfer polynucleic acid is a plasmid or a vector.

In some embodiments, the kit further comprises a small molecule inducer corresponding to a chemically inducible promoter of the engineered cell.

In some embodiments, the kit further comprises an engineered cell comprising a chemically inducible promoter comprising the nucleic acid sequence of one or more of SEQ ID NOs: 1-9 operably linked to the nucleic acid sequence encoding for Gag, the nucleic acid sequence encoding for Pol, the nucleic acid sequence encoding for VSV-G, the nucleic acid sequence encoding for Rev, or a combination thereof. In some embodiments, the nucleic acid sequence encoding for Gag, the nucleic acid sequence encoding for Pol, the nucleic acid sequence encoding for VSV-G, and the nucleic acid sequence encoding for Rev are each operably linked to a chemically inducible promoter comprising the nucleic acid sequence of one or more of SEQ ID NOs: 1-9.

In some embodiments, the kit comprises an engineered cell comprising at least two chemically inducible promoters. In some embodiments, the kit comprises an engineered cell comprises at least two chemically inducible promoters, wherein two or more of the at least two chemically inducible promoters are distinct.

In some embodiments, the kit comprises a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator. In some embodiments, the kit comprises a transcriptional activator comprising the amino acid sequence of any one of SEQ ID NOs: 10-12. In some embodiments, the kit comprises the small molecule inducer doxycycline or tetracycline.

In some aspects, this application discloses a method of producing a lentiviral vector comprising:
(a) introducing a transfer polynucleic acid into an engineered cell described herein; and
(b) contacting the engineered cell with a small molecule inducer corresponding to a chemically inducible promoter of the engineered cell, thereby inducing expression of Gag, Pol, VSV-G, and Rev; wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell; wherein (b) occurs before, concurrently with, or after (a).

In some embodiments, the engineered cell comprises a chemically inducible promoter comprising the nucleic acid sequence of one or more of SEQ ID NOs: 1-3 operably linked to the nucleic acid sequence encoding for Gag, the nucleic acid sequence encoding for Pol, the nucleic acid sequence encoding for VSV-G, the nucleic acid sequence encoding for Rev, or a combination thereof.

In some embodiments, the engineered cell comprises the nucleic acid sequence encoding for Gag, the nucleic acid sequence encoding for Pol, the nucleic acid sequence encoding for VSV-G, and the nucleic acid sequence encoding for Rev are each operably linked to a chemically inducible promoter comprising the nucleic acid sequence of one or more of SEQ ID NOs: 1-3.

In some embodiments, the engineered cell comprises at least two chemically inducible promoters. In some embodiments, the engineered cell comprises at least two chemically inducible promoters, wherein two or more of the at least two chemically inducible promoters are distinct.

In some embodiments, the engineered cell comprises a transcriptional activator comprising the amino acid sequence of any one of SEQ ID NOs: 10-12.

In some embodiments, the small molecule inducer is doxycycline or tetracycline.

In some aspects this application discloses a method of producing a lentiviral vector comprising:
(a) introducing a transfer polynucleic acid into the engineered cell described herein; and
(b) contacting the engineered cell with a small molecule inducer corresponding to a chemically inducible promoter of the engineered cell, thereby inducing expression of Gag, Pol, VSV-G, and Rev;
wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell;
wherein (b) occurs before, concurrently with, or after (a).

In some embodiments, the engineered cell comprises a chemically inducible promoter comprising the nucleic acid sequence of one or more of SEQ ID NOs: 1-3 operably linked to the nucleic acid sequence encoding for Gag, the nucleic acid sequence encoding for Pol, the nucleic acid sequence encoding for VSV-G, the nucleic acid sequence encoding for Rev, or a combination thereof.

In some embodiments, the nucleic acid sequence encoding for Gag, the nucleic acid sequence encoding for Pol, the nucleic acid sequence encoding for VSV-G, and the nucleic acid sequence encoding for Rev are each operably linked to a chemically inducible promoter comprising the nucleic acid sequence of one or more of SEQ ID NOs: 1-3.

In some embodiments, the engineered cell comprises at least two chemically inducible promoters.

In some embodiments, two or more of the at least two chemically inducible promoters are distinct.

In some embodiments, the transcriptional activator comprises the amino acid sequence of any one of SEQ ID NOs: 10-12.

In some embodiments, the small molecule inducer is doxycycline or tetracycline.

In some aspects this application discloses a method of producing a lentiviral vector in an engineered cell, wherein the engineered cell comprises one or more stably integrated heterologous polynucleic acids collectively comprising: a nucleic acid sequence encoding for Gag; a nucleic acid sequence encoding for Pol; a nucleic acid sequence encoding for VSV-G; and a nucleic acid encoding for Rev; at least one of which is operably linked to an exogenous promoter that is capable of being bound by an exogenous transcriptional activator in the absence of a small molecule repressor, said method comprising:
(a) introducing a transfer polynucleic acid into the engineered cell; and
(b) introducing the exogenous transcriptional activator into the engineered cell; and
(c) culturing the cell in the absence of the small molecule repressor, thereby inducing expression of Gag, Pol, VSV-G, and Rev;
wherein (b) occurs before, concurrently with, or after (a).

In some embodiments, the introducing in (b) is performed in the presence of the small molecule repressor.

In some embodiments, the introducing in (b) is performed by: introducing a heterologous polynucleic acid into the cell, wherein the heterologous polynucleic acid comprises a nucleic acid sequence of the exogenous transcriptional activator operably linked to a nucleic acid of a promoter; and expressing the exogenous transcriptional activator.

In some embodiments, the exogenous promoter comprises the nucleic acid sequence of one or more of SEQ ID NOs: 1-3.

In some embodiments, the nucleic acid sequence encoding for Gag, the nucleic acid sequence encoding for Pol, the nucleic acid sequence encoding for VSV-G, and the nucleic acid sequence encoding for Rev are each operably linked to an exogenous promoter comprising the nucleic acid sequence of one or more of SEQ ID NOs: 1-3.

In some embodiments, the engineered cell comprises at least two exogenous promoters.

In some embodiments, two or more of the at least two exogenous promoters are distinct.

In some embodiments, the transcriptional activator is Tetracycline-controlled transactivator (tTA).

In some embodiments, the tTA comprises the amino acid sequence of one or more of SEQ ID NOs: 11 and 27-28.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows an exemplary plasmid schematic for testing dox-inducible lentiviral vector production in transient transfection format. “TetOn” indicates a variant of rtTA, “GP” indicates lentivirus Gag-Pol genes, “VSV-G” indicates Vesicular stomatitis virus G protein, “Rev” indicates lentivirus Rev gene. For lentiviral genes, different combinations of either dox-inducible TRE promoters or constitutive CMV promoters were tested.

FIG. 2 shows an exemplary plasmid schematic for stable integration plasmids for a semi-stable lentiviral vector production system. Selection indicates antibiotic resistance genes used to select for stable integrants. The lentiviral transfer plasmid (not shown in the figure) must be transfected prior to or simultaneously with induction of lentiviral genes, while the other genes are stably integrated into the genome. In addition to the plasmids depicted, alternative plasmids were designed and tested using CMV to drive expression of lentiviral genes as well.

FIG. 3 shows an exemplary plasmid schematic for stable integration of plasmids for complete stable lentiviral vector production system. The complete stable system combines VSV-G and Rev genes on a single plasmid and includes the LTR-containing transfer plasmid to be stably integrated.

FIGS. 4A-4B show results from testing combinations of dox-inducible or constitutive promoters driving lentiviral genes in transient transfection format. FIG. 4A. shows combinations that were tested. FIG. 4B shows the results for samples 1-16. Addition of doxycycline and TetOn to the transfection mix resulted in minimal reduction in lentiviral titers. Placing Gag-Pol, Rev, or VSV-G under a TRE promoter resulted in much decreased titers in absence of doxycycline and near wild type titers in the presence of doxycycline. The fully inducible system does have ˜4.2-fold reduced titers compared to wild type when doxycycline is added, though this reduction may be compensated for by downstream optimization steps.

FIGS. 5A-5B shows results from testing combinations of dox-inducible or constitutive promoters driving lentiviral genes in semi-stable format. FIG. 5A shows the combinations that were tested with or without the presence of Dox. FIG. 5B shows the results of four replicates of each combination/condition (three replicates for negative control). All tested combinations of constitutive or dox-inducible genes were able to induce production of lentivirus in presence of Dox compared to the absence of Dox. Using TRE promoters to drive each of the three genes resulted in among the best inducibility. Greater inducibility could be obtained if Gag-Pol and Rev were transfected instead of being genomically integrated, but that design still maintains the issues associated with scaling up growth volumes.

FIG. 6 shows an exemplary plasmid schematic for a complete, stably integrated lentiviral vector production system. This embodiment of the complete stable system encodes VSV-G, Gag-Pol (“GP”) and Rev genes on separate nucleic acid molecules and includes the LTR-containing transfer plasmid to be stably integrated. VSV-G is encoded on the same nucleic acid molecule as the TetOn element.

FIG. 7 shows an exemplary plasmid schematic for a complete, stably integrated lentiviral vector production system. This embodiment of the complete stable system encodes VSV-G, Gag-Pol (“GP”) and Rev genes on separate nucleic acid molecules and includes the LTR-containing transfer plasmid to be stably integrated. Rev is encoded on the same nucleic acid molecule as the TetOn element.

FIG. 8 shows inducible production of lentivirus using HEK293T cells comprising the stably integrated nucleic acid molecules of FIG. 3. Dox is added at a concentration ranging from 0 nM to 1000 nM. In the absence of doxycycline, the stable producer cells produce minimal infectious titers, while in the presence of greater than 20 nM doxycycline, titers near those achievable with transient transfection. Titers from stable producer titers are approximately 86% those of the traditional transfection samples.

FIG. 9 shows an exemplary plasmid schematic for stable integration of plasmids for a stable lentiviral vector packaging system that does not require doxycycline addition. Tetracycline-controlled transactivator (tTA)—which does not require doxycycline to activate gene expression from the TRE promoter—is provided on the transiently transfected transfer plasmid, rather than using genomically integrated TetOn.

### DETAILED DESCRIPTION

The production of viral vectors normally entails transient transfection of plasmids into cell culture. However, stable integration of genes necessary to produce therapeutic viral vectors into the genome offers several advantages compared to traditional production via transient transfection. Since cells amplify the viral genes during their own cell division, large quantities of DNA and transfection reagent no longer need to be procured for the transfection process, reducing costs. Also, since the DNA is already within the nucleus, viral titers may be higher and more consistent due to minimal numbers of “untransfected” cells and reduced variation associated with transfection steps. The simpler production process also saves time and reduces the scale and number of optimization steps needed to achieve high viral titers.

Standard third generation lentivirus packaging and envelope plasmids contain genes that are traditionally driven by constitutive CMV promoters. However, the ability for VSV-G to fuse cells together and halt growth prevents the creation of stable cells producing lentivirus. Potential toxicities associated with Gag-Pol and Rev also complicate creation of producer cells.

Described herein are lentiviral vector production systems. Also described herein are kits comprising a lentiviral vector production system. Finally, engineered cells comprising a lentiviral vector production system (or at least a portion thereof) are described, as well as methods of using the engineered cells for lentiviral vector production.

### I. Lentiviral Vector Production Systems

In some aspects, the disclosure relates to lentiviral vector production systems. A lentiviral vector production system, as described herein, comprises one or more polynucleic acids that collectively encode the gene products required for generation of a lentiviral vector in a recombinant host cell (or an “engineered cell” as described herein). The lentiviral vector production systems described herein comprise one or more polynucleotides that collectively encode for lentiviral gene products Gag (or a functional variant thereof), Pol (or a functional variant thereof), VSV-G (or a functional variant thereof), and Rev (or a functional variant thereof).

In some embodiments, a lentiviral vector production system is (i.e., the gene products of the viral vector component are) encoded on a single polynucleic acid. In other embodiments, multiple polynucleic acids collectively comprise a lentiviral vector production system (i.e., at least two of the gene products of the viral vector component are encoded on different polynucleic acids). For example, a lentiviral vector production system may comprise at least 2, at least 3, at least 4, or at least 5 polynucleic acids. In some embodiments, a lentiviral vector production system comprises 2, 3, 4, or 5 polynucleic acids. Exemplary lentiviral production system architectures are provided below (Part IC).

Gag is a precursor structural protein of the lentiviral particle containing matrix, capsid, and nucleocapsid components. In some embodiments, a lentiviral vector production system comprises a polynucleic acid encoding for Gag. In some embodiments, Gag comprises the amino acid sequence of SEQ ID NO: 17. In some embodiments, a lentiviral vector production system comprises a polynucleic acid encoding for a functional variant of Gag. A functional variant of Gag comprises at least 80% identity (at least 85%, at least 90%, at least 95%, at least 96%, at least 97%, at least 98%, or at least 99% identity) with SEQ ID NO: 17 and maintains at least 80% of the function (at least 85%, at least 90%, at least 95%, at least 96%, at least 97%, at least 98%, or at least 99% of the function) of Gag (that is, its structural function).

Methods of determining the extent of identity between two sequences (e.g., two amino acid sequences or two polynucleic acids) are known to those having ordinary skill in the art. One exemplary method is the use of Basic Local Alignment Search Tool (BLAST®) software with default parameters (blast.ncbi.nlm.nih.gov/Blast.cgi).

Pol is precursor protein containing reverse transcriptase and integrase components. In some embodiments, a lentiviral vector production system comprises a polynucleic acid encoding for Pol. In some embodiments, Pol comprises the amino acid sequence of SEQ ID NO: 18. In some embodiments, a lentiviral vector production system comprises a polynucleic acid encoding for a functional variant of Pol. A functional variant of Pol comprises at least 80% identity (at least 85%, at least 90%, at least 95%, at least 96%, at least 97%, at least 98%, or at least 99% identity) with SEQ ID NO: 18 and maintains at least 80% of the function (at least 85%, at least 90%, at least 95%, at least 96%, at least 97%, at least 98%, or at least 99% of the function) of Pol (that is, its function as a reverse transcriptase and an integrase).

In some embodiments, a lentiviral production system comprises a polynucleic acid encoding for a fusion protein comprising Gag (or a functional variant thereof) and Pol (or a functional variant thereof).

VSV-G is an envelope protein. In some embodiments, a lentiviral vector production system comprises a polynucleic acid encoding for VSV-G. In some embodiments, VSV-G comprises the amino acid sequence of SEQ ID NO: 19. In some embodiments, a lentiviral vector production system comprises a polynucleic acid encoding for a functional variant of VSV-G. A functional variant of VSV-G comprises at least 80% identity (at least 85%, at least 90%, at least 95%, at least 96%, at least 97%, at least 98%, or at least 99% identity) with SEQ ID NO: 19 and maintains at least 80% of the function (at least 85%, at least 90%, at least 95%, at least 96%, at least 97%, at least 98%, or at least 99% of the function) of VSV-G (that is, its function as an envelope protein).

Rev is a transport protein that binds Rev response elements within transcripts to facilitate their nuclear export. In some embodiments, a lentiviral vector production system comprises a polynucleic acid encoding for Rev. In some embodiments, Rev comprises the amino acid sequence of SEQ ID NO: 20. In some embodiments, a lentiviral vector production system comprises a polynucleic acid encoding for a functional variant of Rev. A functional variant of Rev comprises at least 80% identity (at least 85%, at least 90%, at least 95%, at least 96%, at least 97%, at least 98%, or at least 99% identity) with SEQ ID NO: 20 and maintains at least 80% of the function (at least 85%, at least 90%, at least 95%, at least 96%, at least 97%, at least 98%, or at least 99% of the function) of Rev (that is, its function as a transport protein).

The lentiviral production systems described herein comprise at least one expression cassette. As used herein, the term “expression cassette” refers to a polynucleic acid sequence encoding a nucleic acid sequence of a promoter that is operably linked to a nucleic acid encoding a product (e.g., an RNA product(s) and/or a polypeptide product(s), such as Gag (or a functional variant thereof), Pol (or a functional variant thereof), VSV-G (or a functional variant thereof), Rev (or a functional variant thereof), or any combination thereof). In some embodiments, multiple products are encoded within a single expression cassette. For example, in some embodiments, a single promoter drives expression of a polycistronic RNA encoding for multiple products (an RNA product(s) and/or a polypeptide product(s)). A polycistronic RNA may comprises a nucleic acid sequence of an internal ribosomal entry site (IRES) and/or a nucleic acid sequence of a viral 2A peptide (V2A).

An IRES may comprises the nucleic acid
sequence of SEQ ID NO: 21:
CCCCTCTCCCTCCCCCCCCCCTAACGTTACTGGCCGAAGCCGCTT
GGAATAAGGCCGGTGTGCGTTTGTCTATATGTTATTTTCCACCAT
ATTGCCGTCTTTTGGCAATGTGAGGGCCCGGAAACCTGGCCCTGT
CTTCTTGACGAGCATTCCTAGGGGTCTTTCCCCTCTCGCCAAAGG
AATGCAAGGTCTGTTGAATGTCGTGAAGGAAGCAGTTCCTCTGGA
AGCTTCTTGAAGACAAACAACGTCTGTAGCGACCCTTTGCAGGCA
GCGGAACCCCCCACCTGGCGACAGGTGCCTCTGCGGCCAAAAGCC
ACGTGTATAAGATACACCTGCAAAGGCGGCACAACCCCAGTGCCA
CGTTGTGAGTTGGATAGTTGTGGAAAGAGTCAAATGGCTCTCCTC
AAGCGTATTCAACAAGGGGCTGAAGGATGCCCAGAAGGTACCCCA
TTGTATGGGATCTGATCTGGGGCCTCGGTGCACATGCTTTACATG
TGTTTAGTCGAGGTTAAAAAAACGTCTAGGCCCCCCGAACCACGG
GGACGTGGTTTTCCTTTGAAAAACACGATGATAATATG
An IRES may comprise the nucleic acid
sequence of SEQ ID NO: 22:
CCCCCCCCCCTAACGTTACTGGCCGAAGCCGCTTGGAATAAGGCC
GGTGTGCGTTTGTCTATATGTTATTTTCCACCATATTGCCGTCTT
TTGGCAATGTGAGGGCCCGGAAACCTGGCCCTGTCTTCTTGACGA
GCATTCCTAGGGGTCTTTCCCCTCTCGCCAAAGGAATGCAAGGTC
TGTTGAATGTCGTGAAGGAAGCAGTTCCTCTGGAAGCTTCTTGAA
GACAAACAACGTCTGTAGCGACCCTTTGCAGGCAGCGGAACCCCC
CACCTGGCGACAGGTGCCTCTGCGGCCAAAAGCCACGTGTATAAG
ATACACCTGCAAAGGCGGCACAACCCCAGTGCCACGTTGTGAGTT
GGATAGTTGTGGAAAGAGTCAAATGGCTCTCCTCAAGCGTATTCA
ACAAGGGGCTGAAGGATGCCCAGAAGGTACCCCATTGTATGGGAT
CTGATCTGGGGCCTCGGTGCACATGCTTTACATGTGTTTAGTCGA
GGTTAAAAAAACGTCTAGGCCCCCCGAACCACGGGGACGTGGTTT
TCCTTTGAAAAACACGATGATAATAGTTATC

A viral 2A peptide may comprise the amino acid sequence of ATNFSLLKQAGDVEENPGP (SEQ ID NO: 23), EGRGSLLTCGDVEENPGP (SEQ ID NO: 24), QCTNYALLKLAGDVESNPGP (SEQ ID NO: 25), or VKQTLNFDLLKLAGDVESNPGP (SEQ ID NO: 26).

As described herein, a promoter is “operably linked” to a nucleic acid coding sequence when the position of the promoter relative to the nucleic acid coding sequence is such that binding of a transcriptional activator to the promoter can induce expression of the coding sequence. A promoter of an expression cassette may be a constitutive promoter or an inducible promoter.

A promoter may be a constitutive promoter (i.e., an unregulated promoter that allows for continual transcription). Examples of constitutive promoters are known in the art and include, but are not limited to, cytomegalovirus (CMV) promoters, elongation factor 1 α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July, 110(28): 11284-89; Pub. No.: US 2014/377861 A1; Qin et al. PloS One. 2010 May; 5(5): e10611.—the entireties of which are incorporated herein by reference.

Alternatively, a promoter may be an inducible promoter (i.e., only activates transcription under specific circumstances). An inducible promoter may be, for example, a chemically inducible promoter, a temperature inducible promoter, or a light inducible promoter. Examples of chemically inducible promoters are known in the art and include, but are not limited to, tetracycline/doxycycline inducible promoters, cumate inducible promoters, ABA inducible promoters, CRY2-CIB1 inducible promoters, DAPG inducible promoters, mifepristone inducible promoters, lactose inducible promotors, and arabinose inducible promoters. See e.g., Stanton et al., ACS Synth. Biol. 2014 Dec. 19; 3(12): 880-91; Liang et al., Sci. Signal. 2011 Mar. 15; 4(164): rs2; Das et al., Curr. Gene. Ther. 2016; 16(3): 156-167; U.S. Pat. No. 7,745,592 B2; U.S. Pat. No. 7,935,788 B2—the entireties of which are incorporated herein by reference. A chemically inducible promoter may comprise the nucleic acid sequence of one or more of SEQ ID NOs: 1-9.

### A. Selectable Markers

As described above, a lentiviral vector production system, as described herein, comprises one or more polynucleic acids that collectively encode the gene products required for generation of a lentiviral vector in a recombinant host cell (or an “engineered cell” as described herein). In some embodiments, one or more of the polynucleic acids of a lentiviral vector production system comprises an expression cassette comprising: (i) a nucleic acid sequence of a promoter (constitutive or inducible, as described herein); and (ii) a nucleic acid sequence encoding a selectable maker. In some embodiments, each of the polynucleic acids of a viral production system comprises a selectable maker. In some embodiments, each polynucleic acid of a lentiviral production system comprises a nucleic acid sequence of a distinct selectable marker.

As used herein, the term “selectable marker” refers to a protein that—when introduced into or expressed in a cell—confers a trait that is suitable for selection.

A selectable marker may be a fluorescent protein. Examples of fluorescent proteins are known in the art (e.g., TagBFP, EBFP2, EGFP, EYFP, RFP, mKO2, or Sirius). See e.g., U.S. Pat. No. 5,874,304; EP 0969284 A1; Pub. No.: US 2010/167394 A—the entireties of which are incorporated here by reference.

Alternatively, or in addition, a selectable marker may be an antibiotic resistance protein. Examples of antibiotic resistance proteins are known in the art (e.g., facilitating puromycin, hygromycin, neomycin, zeomycin, blasticidin, or phleomycin selection). See e.g., Pub. No.: WO 1997/15668 A2; Pub. No.: WO 1997/43900 A1—the entireties of which are incorporated here by reference.

### B. Inducible Lentiviral Vector Production Systems

In some embodiments, a lentiviral vector production systems described herein comprises one or more polynucleotides that collectively encode for lentiviral gene products Gag (or a functional variant thereof), Pol (or a functional variant thereof), VSV-G (or a functional variant thereof), and Rev (or a functional variant thereof), at least one of which is operably linked to a chemically inducible promoter.

In any of the embodiments described in this section, a chemically inducible promoter may comprise a tetracycline/doxycycline inducible promoter, a cumate inducible promoter, a ABA inducible promoter, a CRY2-CIB1 inducible promoter, a DAPG inducible promoter, a mifepristone inducible promoter, or a combination thereof. In some embodiments, a chemically inducible promoter comprises the nucleic acid sequence of one or more of SEQ ID NOs: 1-9. In some embodiments, a chemically inducible promoter comprises the nucleic acid sequence of SEQ ID NO: 1. In some embodiments, a chemically inducible promoter comprises the nucleic acid sequence of SEQ ID NO: 2. In some embodiments, a chemically inducible promoter comprises the nucleic acid sequence of SEQ ID NO: 3. In some embodiments, a chemically inducible promoter comprises the nucleic acid sequence of SEQ ID NO: 4. In some embodiments, a chemically inducible promoter comprises the nucleic acid sequence of SEQ ID NO: 5. In some embodiments, a chemically inducible promoter comprises the nucleic acid sequence of SEQ ID NO: 6. In some embodiments, a chemically inducible promoter comprises the nucleic acid sequence of SEQ ID NO: 7. In some embodiments, a chemically inducible promoter comprises the nucleic acid sequence of SEQ ID NO: 8. In some embodiments, a chemically inducible promoter comprises the nucleic acid sequence of SEQ ID NO: 9.

In some embodiments, the nucleic acid sequence encoding Gag (or a functional variant thereof) is operably linked to a chemically inducible promoter (as described herein); optionally wherein: the nucleic acid sequence encoding Pol (or a functional variant thereof) is operably linked to a chemically inducible promoter (as described herein); and/or the nucleic acid sequence encoding VSV-G (or a functional variant thereof) is operably linked to a chemically inducible promoter (as described herein); and/or the nucleic acid sequence encoding Rev (or a functional variant thereof) is operably linked to a chemically inducible promoter (as described herein).

In some embodiments, the nucleic acid sequence encoding Pol (or a functional variant thereof) is operably linked to a chemically inducible promoter (as described herein); optionally wherein: the nucleic acid sequence encoding Gag (or a functional variant thereof) is operably linked to a chemically inducible promoter (as described herein); and/or the nucleic acid sequence encoding VSV-G (or a functional variant thereof) is operably linked to a chemically inducible promoter (as described herein); and/or the nucleic acid sequence encoding Rev (or a functional variant thereof) is operably linked to a chemically inducible promoter (as described herein).

In some embodiments, the nucleic acid sequence encoding VSV-G (or a functional variant thereof) is operably linked to a chemically inducible promoter (as described herein); optionally wherein: the nucleic acid sequence encoding Gag (or a functional variant thereof) is operably linked to a chemically inducible promoter (as described herein); and/or the nucleic acid sequence encoding Pol (or a functional variant thereof) is operably linked to a chemically inducible promoter (as described herein); and/or the nucleic acid sequence encoding Rev (or a functional variant thereof) is operably linked to a chemically inducible promoter (as described herein).

In some embodiments, the nucleic acid sequence encoding Rev (or a functional variant thereof) is operably linked to a chemically inducible promoter (as described herein); optionally wherein: the nucleic acid sequence encoding Gag (or a functional variant thereof) is operably linked to a chemically inducible promoter (as described herein); and/or the nucleic acid sequence encoding Pol (or a functional variant thereof) is operably linked to a chemically inducible promoter (as described herein); and/or the nucleic acid sequence encoding VSV-G (or a functional variant thereof) is operably linked to a chemically inducible promoter (as described herein).

In some embodiments, a lentiviral vector production system described herein comprises one or more polynucleotides that collectively encode for lentiviral gene products Gag (or a functional variant thereof), Pol (or a functional variant thereof), VSV-G (or a functional variant thereof), and Rev (or a functional variant thereof); wherein each is operably linked to a chemically inducible promoter (as described herein).

In some embodiments, a lentiviral vector production system described herein comprises a single chemically inducible promoter. In such embodiments, the single chemically inducible promoter may be operably linked to Gag (or a functional variant thereof), and/or Pol (or a functional variant thereof), and/or VSV-G (or a functional variant thereof), and/or Rev (or a functional variant thereof).

In some embodiments, a lentiviral vector production system comprises at least two, at least three, at least four, or at least five chemically inducible promoters. In some embodiments, a lentiviral vector production system comprises 2, 3, 4, or 5 chemically inducible promoters.

In some embodiments, wherein a lentiviral vector production system comprises at least two chemically inducible promoter, two or more of the chemically inducible promoters comprise the same nucleic acid sequence. In some embodiments, wherein a lentiviral vector production system comprises at least two chemically inducible promoter, each of the chemically inducible promoters comprise the same nucleic acid sequence. In some embodiments, wherein a lentiviral vector production system comprises at least two chemically inducible promoter, one or more of the chemically inducible promoters comprises a distinct nucleic acid sequence. In some embodiments, wherein a lentiviral vector production system comprises at least two chemically inducible promoter, each of the chemically inducible promoters comprises a distinct nucleic acid sequence.

An inducible lentiviral vector production system described herein may further comprise a polynucleic acid comprising an expression cassette comprising; (i) a nucleic acid sequence of a promoter (constitutive or inducible, as described herein); and (ii) a nucleic acid sequence encoding a transcriptional activator, wherein the transcriptional activator, when expressed in the presence of a small molecule inducer, binds to a chemically inducible promoter of the engineered cell. In embodiments wherein a lentiviral vector production system comprises two or more distinct chemically inducible promoters, the system may comprise nucleic acid sequences of two or more corresponding transcriptional activators.

In some embodiments, a transcriptional activator is Tet-On 3G. In some embodiments, Tet-On 3G comprises the amino acid sequence of SEQ ID NO: 10. In some embodiments, a transcriptional activator is a functional variant of Tet-On 3G. A functional variant of Tet-On 3G comprises at least 80% identity (at least 85%, at least 90%, at least 95%, at least 96%, at least 97%, at least 98%, or at least 99% identity) with SEQ ID NO: 10 and maintains at least 80% of the function (at least 85%, at least 90%, at least 95%, at least 96%, at least 97%, at least 98%, or at least 99% of the function) of Tet-On 3G (that is, its function as a transcriptional activator).

In some embodiments, a transcriptional activator is TetOff-Advanced. In some embodiments, TetOff-Advanced comprises the amino acid sequence of SEQ ID NO: 11. In some embodiments, a transcriptional activator is a functional variant of TetOff-Advanced. A functional variant of TetOff-Advanced comprises at least 80% identity (at least 85%, at least 90%, at least 95%, at least 96%, at least 97%, at least 98%, or at least 99% identity) with SEQ ID NO: 11 and maintains at least 80% of the function (at least 85%, at least 90%, at least 95%, at least 96%, at least 97%, at least 98%, or at least 99% of the function) of TetOff-Advanced (that is, its function as a transcriptional activator).

In some embodiments, a transcriptional activator is VanR-VP16. In some embodiments, VanR-VP16 comprises the amino acid sequence of SEQ ID NO: 12. In some embodiments, a transcriptional activator is a functional variant of VanR-VP16. A functional variant of VanR-VP16 comprises at least 80% identity (at least 85%, at least 90%, at least 95%, at least 96%, at least 97%, at least 98%, or at least 99% identity) with SEQ ID NO: 12 and maintains at least 80% of the function (at least 85%, at least 90%, at least 95%, at least 96%, at least 97%, at least 98%, or at least 99% of the function) of VanR-VP16 (that is, its function as a transcriptional activator).

In some embodiments, a transcriptional activator is TtgR-VP16. In some embodiments, TtgR-VP16 comprises the amino acid sequence of SEQ ID NO: 13. In some embodiments, a transcriptional activator is a functional variant of TtgR-VP16. A functional variant of TtgR-VP16 comprises at least 80% identity (at least 85%, at least 90%, at least 95%, at least 96%, at least 97%, at least 98%, or at least 99% identity) with SEQ ID NO: 13 and maintains at least 80% of the function (at least 85%, at least 90%, at least 95%, at least 96%, at least 97%, at least 98%, or at least 99% of the function) of TtgR-VP16 (that is, its function as a transcriptional activator).

In some embodiments, a transcriptional activator is PhlF-VP16. In some embodiments, PhlF-VP16 comprises the amino acid sequence of SEQ ID NO: 14. In some embodiments, a transcriptional activator is a functional variant of PhlF-VP16. A functional variant of PhlF-VP16 comprises at least 80% identity (at least 85%, at least 90%, at least 95%, at least 96%, at least 97%, at least 98%, or at least 99% identity) with SEQ ID NO: 14 and maintains at least 80% of the function (at least 85%, at least 90%, at least 95%, at least 96%, at least 97%, at least 98%, or at least 99% of the function) of PhlF-VP16 (that is, its function as a transcriptional activator).

In some embodiments, a transcriptional activator is cTA. In some embodiments, cTA comprises the amino acid sequence of SEQ ID NO: 15. In some embodiments, a transcriptional activator is a functional variant of cTA. A functional variant of cTA comprises at least 80% identity (at least 85%, at least 90%, at least 95%, at least 96%, at least 97%, at least 98%, or at least 99% identity) with SEQ ID NO: 15 and maintains at least 80% of the function (at least 85%, at least 90%, at least 95%, at least 96%, at least 97%, at least 98%, or at least 99% of the function) of cTA (that is, its function as a transcriptional activator).

In some embodiments, a transcriptional activator is rcTA. In some embodiments, rcTA comprises the amino acid sequence of SEQ ID NO: 16. In some embodiments, a transcriptional activator is a functional variant of rcTA. A functional variant of rcTA comprises at least 80% identity (at least 85%, at least 90%, at least 95%, at least 96%, at least 97%, at least 98%, or at least 99% identity) with SEQ ID NO: 16 and maintains at least 80% of the function (at least 85%, at least 90%, at least 95%, at least 96%, at least 97%, at least 98%, or at least 99% of the function) of rcTA (that is, its function as a transcriptional activator).

### C. Exemplary Lentiviral Vector Production System Architectures

For exemplary purpose, select lentiviral vector production system architectures are described below.

### 1. First Exemplary Architecture

In some embodiments, a lentiviral vector production system comprises: (a) a first polynucleic acid comprising the nucleic acid sequence encoding for Gag (or a functional variant thereof) and the nucleic acid sequence encoding for Pol (or a functional variant thereof); (b) a second polynucleic acid comprising the nucleic acid sequence encoding for VSV-G (or a functional variant thereof); and (c) a third polynucleic acid comprising the nucleic acid sequence encoding for Rev (or a functional variant thereof).

In some embodiments, the first polynucleic acid comprises a first expression cassette comprising: (i) a nucleic acid sequence of a first chemically inducible promoter (as described herein); and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). In some embodiments, the first chemically inducible promoter comprises the nucleic acid sequence of one or more of SEQ ID NOs: 1-9. In some embodiments, the first polynucleic acid further comprises a nucleic acid sequence of a selection marker (as described herein) that is operably linked to a nucleic acid sequence of a promoter (constitutive or inducible, as described herein).

In some embodiments, the second polynucleic acid comprises a second expression cassette comprising: (i) a nucleic acid sequence of a second chemically inducible promoter (as described herein); and (ii) the nucleic acid sequence encoding for VSV-G (or a functional variant thereof). In some embodiments, the second chemically inducible promoter comprises the nucleic acid sequence of one or more of SEQ ID NOs: 1-9. In some embodiments, the second polynucleic acid further comprises a nucleic acid sequence of a selection marker (as described herein) that is operably linked to a nucleic acid sequence of a promoter (constitutive or inducible, as described herein).

In some embodiments, the third polynucleic acid comprises a third expression cassette comprising: (i) a nucleic acid sequence of a third chemically inducible promoter (as described herein); and (ii) the nucleic acid sequence encoding for Rev (or a functional variant thereof). In some embodiments, the third chemically inducible promoter comprises the nucleic acid sequence of one or more of SEQ ID NOs: 1-9. In some embodiments, the third polynucleic acid further comprises a nucleic acid sequence of a selection marker (as described herein) that is operably linked to a nucleic acid sequence of a promoter (constitutive or inducible, as described herein).

In some embodiments, the first chemically inducible promoter, the second chemically inducible promoter, and the third chemically inducible promoter comprise the same nucleic acid sequence.

In some embodiments, the lentiviral vector production system further comprises an additional polynucleic acid comprising a nucleic acid sequence of a transcriptional activator operably linked to a nucleic acid sequence of a promoter (constitutive or inducible, as described herein), wherein the transcriptional activator, when expressed in the presence of a small molecule inducer, binds to a chemically inducible promoter of the lentiviral vector production system. In some embodiments, the transcriptional activator comprises the amino acid sequence of any one of SEQ ID NOs: 10-16.

In some embodiments, the first polynucleic acid, the second polynucleic acid, or the third polynucleic acid comprises the additional polynucleic acid. In other embodiments, the additional polynucleic acid is a fourth polynucleic acid of the lentiviral vector production system.

### 2. Second Exemplary Architecture

In some embodiments, a lentiviral vector production system comprises: (a) a first polynucleic acid comprising the nucleic acid sequence encoding for Gag (or a functional variant thereof) and the nucleic acid sequence encoding for Pol (or a functional variant thereof); and (b) a second polynucleic acid comprising the nucleic acid sequence encoding for VSV-G (or a functional variant thereof) and the nucleic acid sequence encoding for Rev (or a functional variant thereof).

In some embodiments, the first polynucleic acid comprises a first expression cassette comprising: (i) a nucleic acid sequence of a first chemically inducible promoter (as described herein); and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). In some embodiments, the first chemically inducible promoter comprises the nucleic acid sequence of one or more of SEQ ID NOs: 1-9. In some embodiments, the first polynucleic acid further comprises a nucleic acid sequence of a selection marker (as described herein) that is operably linked to a nucleic acid sequence of a promoter (constitutive or inducible, as described herein).

In some embodiments, the second polynucleic acid comprises: a second expression cassette comprising: (i) a nucleic acid sequence of a second chemically inducible promoter (as described herein); and (ii) the nucleic acid sequence encoding for VSV-G (or a functional variant thereof); and a third expression cassette comprising: (i) a nucleic acid sequence of a third chemically inducible promoter (as described herein); and (ii) the nucleic acid sequence encoding for Rev (or a functional variant thereof). In some embodiments, the second promoter comprises the nucleic acid sequence of one or more of SEQ ID NOs: 1-9; the third promoter comprises the nucleic acid sequence of one or more of SEQ ID NOs: 1-9; or a combination thereof. In some embodiments, the second polynucleic acid further comprises a nucleic acid sequence of a selection marker (as described herein) that is operably linked to a nucleic acid sequence of a promoter (constitutive or inducible, as described herein).

In some embodiments, the lentiviral vector production system further comprises an additional polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the lentiviral vector production system. In some embodiments, the transcriptional activator comprises the amino acid sequence of one or more of any one of SEQ ID NOs: 10-16.

In some embodiments, the first polynucleic acid or the second polynucleic acid comprises the additional polynucleic acid. In other embodiments, the additional polynucleic acid is a third polynucleic acid of the lentiviral vector production system.

### 3. Third Exemplary Architecture

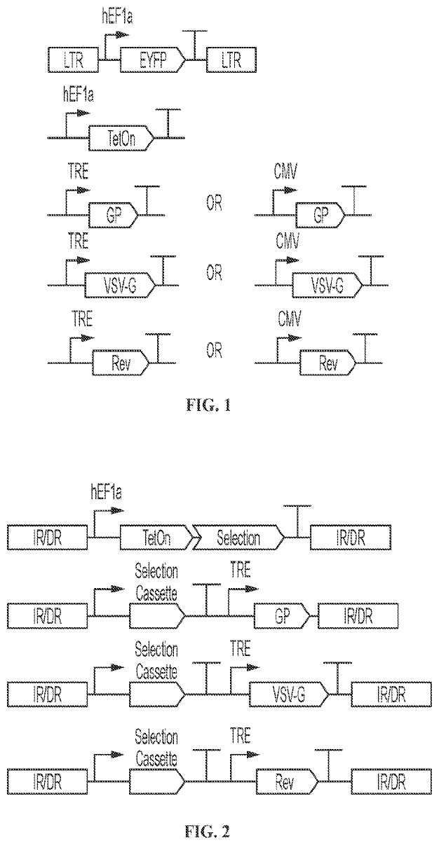

In some embodiments, a lentiviral vector production system architecture is as depicted in FIG. 1.

### 4. Fourth Exemplary Architecture

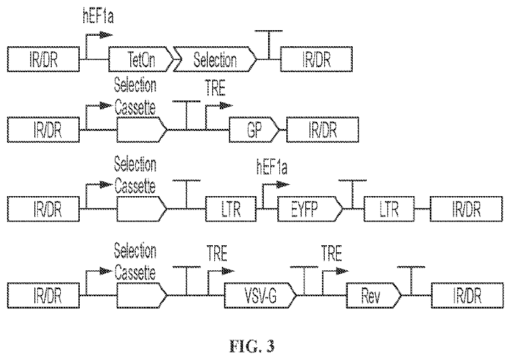

In some embodiments, a lentiviral vector production system architecture is as depicted in FIG. 2.

### 5. Fifth Exemplary Architecture

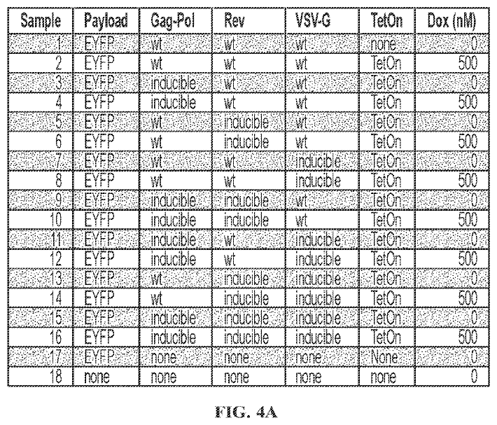

In some embodiments, a lentiviral vector production system architecture is as depicted in FIG. 3.

### 6. Sixth Exemplary Architecture

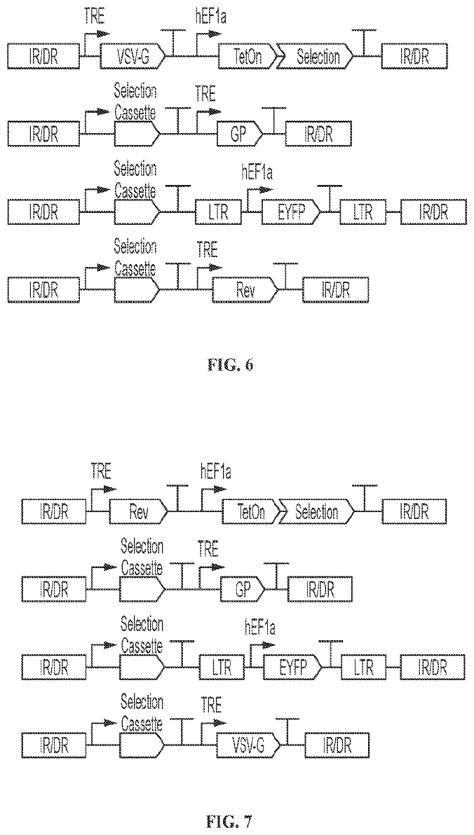

In some embodiments, a lentiviral vector production system architecture is as depicted in FIG. 6.

### 7. Seventh Exemplary Architecture

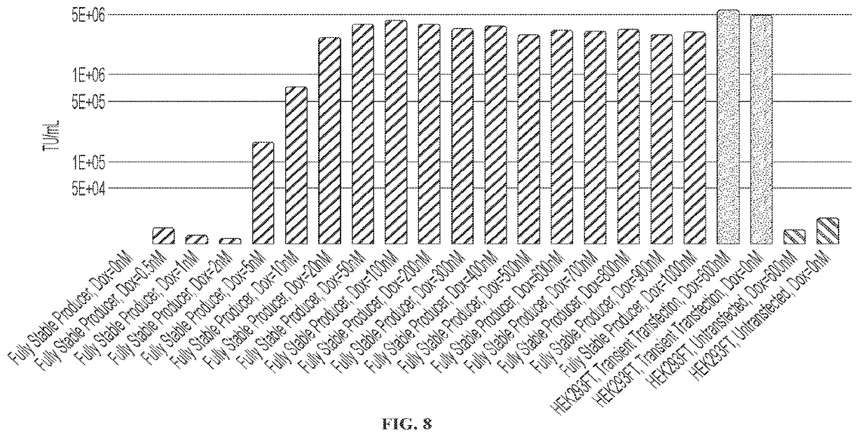

In some embodiments, a lentiviral vector production system architecture is as depicted in FIG. 7.

### 8. Eighth Exemplary Architecture

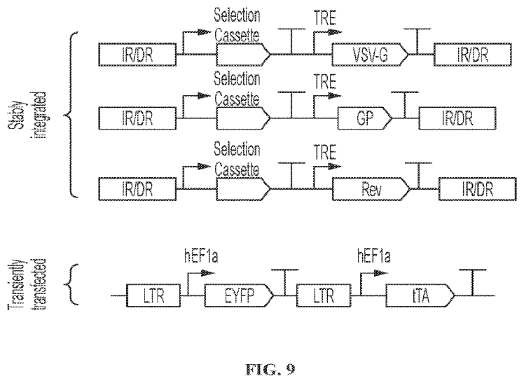

In some embodiments, a lentiviral vector production system architecture is as depicted in FIG. 9.

### II. Engineered Cells

In some aspects, the disclosure relates to engineered cells comprising one or more polynucleic acid of a lentiviral vector production system described in Part I. In the context of an engineered cell, such a polynucleic acid is considered a heterologous polynucleic acid (i.e., a polynucleic acid sequence that is not found in the cell naturally). In some embodiments, an engineered cell comprises each of the polynucleic acids of a lentiviral production system described in Part I.

In some embodiments, a polynucleic acid of a lentiviral vector production system is transiently present in an engineered cell.

In other embodiments, a polynucleic acid of a lentiviral vector production system is stably integrated into the genome of the engineered cell. In some embodiments, each polynucleic acid of a lentiviral vector production system is stably integrated into the genome of the engineered cell. In some embodiments, each polynucleic acid of a lentiviral vector production system is stably integrated at a different position within the genome of the engineered cell.

In some embodiments, a polynucleic acid of a lentiviral vector production system is stably integrated into the genome of the engineered cell at a copy number of at least 5 (e.g., at least 10, at least 15, at least 20, at least 30, at least 40, at least 50, at least 60, at least 70, at least 80, at least 90, at least 100, at least 125, at least 150, at least 175, or at least 200) copies. In some embodiments, a polynucleic acid of a lentiviral vector production system is stably integrated into the genome of the engineered cell at a copy number of 10-20, 10-50, 25-50, 25-75, 25-100, 25-150, 50-75, 50-100, 50-125, 50-150, 50-200, 75-100, 75-125, 75-150, 75-175, 75-200, 100-125, 100-150, 100-175, or 100-200 copies. In some embodiments, a polynucleic acid of a lentiviral vector production system is stably integrated into the genome of the engineered cell at a copy number of 50-75, 50-100, 50-125, 50-150, 50-200, 75-100, 75-125, 75-150, 75-175, 75-200, 100-125, 100-150, 100-175, or 100-200 copies.

In some embodiments, each polynucleic acid of a lentiviral vector production system that is stably integrated into the genome of the engineered cell is stably integrated at a copy number of at least 5 (e.g., at least 10, at least 15, at least 20, at least 30, at least 40, at least 50, at least 60, at least 70, at least 80, at least 90, at least 100, at least 125, at least 150, at least 175, or at least 200) copies. In some embodiments, each polynucleic acid of a lentiviral vector production system that is stably integrated into the genome of the engineered cell is stably integrated at a copy number of 10-20, 10-50, 25-50, 25-75, 25-100, 25-150, 50-75, 50-100, 50-125, 50-150, 50-200, 75-100, 75-125, 75-150, 75-175, 75-200, 100-125, 100-150, 100-175, or 100-200 copies. In some embodiments, each polynucleic acid of a lentiviral vector production system that is stably integrated into the genome of the engineered cell is stably integrated at a copy number of 50-75, 50-100, 50-125, 50-150, 50-200, 75-100, 75-125, 75-150, 75-175, 75-200, 100-125, 100-150, 100-175, or 100-200 copies.

In some embodiments, an engineered cell is derived from a HEK293 cell.

In some embodiments, an engineered cell is derived from a HeLa cell.

In some embodiments, an engineered cell is derived from a BHK cell.

In some embodiments, an engineered cell is derived from a Sf9 cell.

### A. Landing Pad

An engineered cell described herein may further comprise a landing pad. As used herein, the term “landing pad” refers to a heterologous polynucleic acid sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46(8): 4072-4086; U.S. Pat. No. 8,980,579 B2; U.S. Pat. No. 10,017,786 B2; U.S. Pat. No. 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, CCR5, and LiPS-A3S.

In some embodiments, the engineered cell is derived from a CHO cell. In some embodiments, the engineered CHO cell comprises a landing pad that is integrated at a safe harbor locus selected from the group consisting of ROSA26, COSMIC, and H11.

Each of the landing pads described herein comprises at least one recombination site. Recombination sites for various integrases have been identified previously. For example, a landing pad may comprise recombination sites corresponding to a Bxb1 integrase, lambda-integrase, Cre recombinase, Flp recombinase, gamma-delta resolvase, Tn3 resolvase, QC31 integrase, or R4 integrase. Exemplary recombination site sequences are known in the art (e.g., attP, attB, attR, attL, Lox, and Frt).

The landing pads described herein may comprise one or more expression cassettes.

### B. Exemplary Engineered Cells

For exemplary purpose, select engineered cells are described below.

### 1. First Exemplary Engineered Cell

In some embodiments, an engineered cell comprises one or more stably integrated heterologous polynucleic acids collectively comprising: a nucleic acid sequence encoding for Gag (or a functional variant thereof); a nucleic acid sequence encoding for Pol (or a functional variant thereof); a nucleic acid sequence encoding for VSV-G (or a functional variant thereof); and a nucleic acid encoding for Rev (or a functional variant thereof); at least one of which is operably linked to a chemically inducible promoter (as described, for example, in Part I). In some embodiments, the chemically inducible promoter comprises the nucleic acid sequence of one or more of SEQ ID NOs: 1-9.

In some embodiments, the engineered cell comprises a nucleic acid sequence encoding for Gag (or a functional variant thereof) operably linked to a chemically inducible promoter (as described, for example, in Part I). In some embodiments, Gag comprises the amino acid sequence of SEQ ID NO: 17.

In some embodiments, the engineered cell comprises a nucleic acid sequence encoding for Pol (or a functional variant thereof) operably linked to a chemically inducible promoter (as described, for example, in Part I). In some embodiments, Pol (or a functional variant thereof) comprises the amino acid sequence of SEQ ID NO: 18.

In some embodiments, the engineered cell comprises a nucleic acid sequence encoding for VSV-G (or a functional variant thereof) operably linked to a chemically inducible promoter (as described, for example, in Part I). In some embodiments, VSV-G comprises the amino acid sequence of SEQ ID NO: 19.

In some embodiments, the engineered cell comprises a nucleic acid sequence encoding for Rev (or a functional variant thereof) operably linked to a chemically inducible promoter (as described, for example, in Part I). In some embodiments, Rev comprises the amino acid sequence of SEQ ID NO: 20.

In some embodiments, each polynucleic acid of a lentiviral vector production system that is stably integrated into the genome of the engineered cell is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the engineered cell further comprises a stable landing pad (as described herein).

In some embodiments, the engineered cell is derived from a HEK293 cell, a HeLa cell, a BHK cell, or a Sf9 cell.

### 2. Second Exemplary Engineered Cell

In some embodiments, an engineered cell comprises: (a) a first stably integrated heterologous polynucleic acid comprising the nucleic acid sequence encoding for Gag and the nucleic acid sequence encoding for Pol; (b) a second stably integrated heterologous polynucleic acid comprising the nucleic acid sequence encoding for VSV-G; and (c) a third stably integrated heterologous polynucleic acid comprising the nucleic acid sequence encoding for Rev.

In some embodiments, the first stably integrated heterologous polynucleic acid comprises a first expression cassette comprising: (i) a nucleic acid sequence of a first chemically inducible promoter (as described herein); and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). In some embodiments, the first chemically inducible promoter comprises the nucleic acid sequence of one or more of SEQ ID NOs: 1-9. In some embodiments, the first stably integrated heterologous polynucleic acid further comprises a nucleic acid sequence of a selection marker (as described herein) that is operably linked to a nucleic acid sequence of a promoter (constitutive or inducible, as described herein).

In some embodiments, the second stably integrated heterologous polynucleic acid comprises a second expression cassette comprising: (i) a nucleic acid sequence of a second chemically inducible promoter (as described herein); and (ii) the nucleic acid sequence encoding for VSV-G (or a functional variant thereof). In some embodiments, the second chemically inducible promoter comprises the nucleic acid sequence of one or more of SEQ ID NOs: 1-9. In some embodiments, the second stably integrated heterologous polynucleic acid further comprises a nucleic acid sequence of a selection marker (as described herein) that is operably linked to a nucleic acid sequence of a promoter (constitutive or inducible, as described herein).

In some embodiments, the third stably integrated heterologous polynucleic acid comprises a third expression cassette comprising: (i) a nucleic acid sequence of a third chemically inducible promoter (as described herein); and (ii) the nucleic acid sequence encoding for Rev (or a functional variant thereof). In some embodiments, the third chemically inducible promoter comprises the nucleic acid sequence of one or more of SEQ ID NOs: 1-9. In some embodiments, the third stably integrated heterologous polynucleic acid further comprises a nucleic acid sequence of a selection marker (as described herein) that is operably linked to a nucleic acid sequence of a promoter (constitutive or inducible, as described herein).

In some embodiments, the first chemically inducible promoter, the second chemically inducible promoter, and the third chemically inducible promoter comprise the same nucleic acid sequence.

In some embodiments, the engineered cell further comprises an additional polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the lentiviral vector production system. In some embodiments, the transcriptional activator comprises the amino acid sequence of any one of SEQ ID NOs: 10-16.

In some embodiments, the first stably integrated polynucleic acid, the second stably integrated polynucleic acid, or the third stably integrated polynucleic acid comprises the additional polynucleic acid. In other embodiments, the additional polynucleic acid is a fourth stably integrated heterologous polynucleic acid of the engineered cell.

In some embodiments, each polynucleic acid of a lentiviral vector production system that is stably integrated into the genome of the engineered cell is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the engineered cell further comprises a stable landing pad (as described herein).

In some embodiments, the engineered cell is derived from a HEK293 cell, a HeLa cell, a BHK cell, or a Sf9 cell.

### 3. Third Exemplary Engineered Cell

In some embodiments, an engineered cell comprises: (a) a first stably integrated polynucleic acid comprising the nucleic acid sequence encoding for Gag (or a functional variant thereof) and the nucleic acid sequence encoding for Pol (or a functional variant thereof); and (b) a second stably integrated polynucleic acid comprising the nucleic acid sequence encoding for VSV-G (or a functional variant thereof) and the nucleic acid sequence encoding for Rev (or a functional variant thereof).

In some embodiments, the first stably integrated polynucleic acid comprises a first expression cassette comprising: (i) a nucleic acid sequence of a first chemically inducible promoter (as described herein); and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). In some embodiments, the first chemically inducible promoter comprises the nucleic acid sequence of one or more of SEQ ID NOs: 1-9. In some embodiments, the first stably integrated polynucleic acid further comprises a nucleic acid sequence of a selection marker (as described herein) that is operably linked to a nucleic acid sequence of a promoter (constitutive or inducible, as described herein).

In some embodiments, the second stably integrated polynucleic acid comprises: a second expression cassette comprising: (i) a nucleic acid sequence of a second chemically inducible promoter (as described herein); and (ii) the nucleic acid sequence encoding for VSV-G (or a functional variant thereof); and a third expression cassette comprising: (i) a nucleic acid sequence of a third chemically inducible promoter (as described herein); and (ii) the nucleic acid sequence encoding for Rev (or a functional variant thereof). In some embodiments, the second promoter comprises the nucleic acid sequence of one or more of SEQ ID NOs: 1-9; the third promoter comprises the nucleic acid sequence of one or more of SEQ ID NOs: 1-9; or a combination thereof. In some embodiments, the second stably integrated polynucleic acid further comprises a nucleic acid sequence of a selection marker (as described herein) that is operably linked to a nucleic acid sequence of a promoter (constitutive or inducible, as described herein).

In some embodiments, the engineered cell further comprises an additional polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the lentiviral vector production system. In some embodiments, the transcriptional activator comprises the amino acid sequence of one or more of any one of SEQ ID NOs: 10-16.

In some embodiments, the first stably integrated polynucleic acid or the second stably integrated polynucleic acid comprises the additional polynucleic acid. In other embodiments, the additional polynucleic acid is a third stably integrated polynucleic acid of the lentiviral vector production system.

In some embodiments, each polynucleic acid of a lentiviral vector production system that is stably integrated into the genome of the engineered cell is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the engineered cell further comprises a stable landing pad (as described herein).

In some embodiments, the engineered cell is derived from a HEK293 cell, a HeLa cell, a BHK cell, or a Sf9 cell.

### 4. Fourth Exemplary Engineered Cell

In some embodiments, an engineered cell comprises: (a) a first stably integrated heterologous polynucleic acid comprising the nucleic acid sequence encoding for Gag and the nucleic acid sequence encoding for Pol; (b) a second stably integrated heterologous polynucleic acid comprising the nucleic acid sequence encoding for VSV-G; and (c) a third stably integrated heterologous polynucleic acid comprising the nucleic acid sequence encoding for Rev.

In some embodiments, the first stably integrated heterologous polynucleic acid comprises a first expression cassette comprising: (i) a nucleic acid sequence of a first chemically inducible promoter (as described herein); and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). In some embodiments, the first chemically inducible promoter comprises the nucleic acid sequence of one or more of SEQ ID NOs: 1-9. In some embodiments, the first stably integrated heterologous polynucleic acid further comprises a nucleic acid sequence of a selection marker (as described herein) that is operably linked to a nucleic acid sequence of a promoter (constitutive or inducible, as described herein).

In some embodiments, the second stably integrated heterologous polynucleic acid comprises a second expression cassette comprising: (i) a nucleic acid sequence of a second chemically inducible promoter (as described herein); and (ii) the nucleic acid sequence encoding for VSV-G (or a functional variant thereof). In some embodiments, the second chemically inducible promoter comprises the nucleic acid sequence of one or more of SEQ ID NOs: 1-9. In some embodiments, the second stably integrated heterologous polynucleic acid further comprises a nucleic acid sequence of a selection marker (as described herein) that is operably linked to a nucleic acid sequence of a promoter (constitutive or inducible, as described herein). In some embodiments, the second stably integrated heterologous polynucleic acid comprises a nucleic acid sequence of a transcriptional activator operably linked to a nucleic acid sequence of a promoter, wherein the transcriptional activator, when expressed in the presence of a small molecule inducer, binds to a chemically inducible promoter of the lentiviral vector production system. In some embodiments, the transcriptional activator comprises the amino acid sequence of any one of SEQ ID NOs: 10-16.

In some embodiments, the third stably integrated heterologous polynucleic acid comprises a third expression cassette comprising: (i) a nucleic acid sequence of a third chemically inducible promoter (as described herein); and (ii) the nucleic acid sequence encoding for Rev (or a functional variant thereof). In some embodiments, the third chemically inducible promoter comprises the nucleic acid sequence of one or more of SEQ ID NOs: 1-9. In some embodiments, the third stably integrated heterologous polynucleic acid further comprises a nucleic acid sequence of a selection marker (as described herein) that is operably linked to a nucleic acid sequence of a promoter (constitutive or inducible, as described herein).

In some embodiments, the first chemically inducible promoter, the second chemically inducible promoter, and the third chemically inducible promoter comprise the same nucleic acid sequence.

In some embodiments, the first stably integrated polynucleic acid, the second stably integrated polynucleic acid, or the third stably integrated polynucleic acid comprises the additional polynucleic acid. In other embodiments, the additional polynucleic acid is a fourth stably integrated heterologous polynucleic acid of the engineered cell.

In some embodiments, each polynucleic acid of a lentiviral vector production system that is stably integrated into the genome of the engineered cell is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the engineered cell further comprises a stable landing pad (as described herein).

In some embodiments, the engineered cell is derived from a HEK293 cell, a HeLa cell, a BHK cell, or a Sf9 cell.

### 5. Fifth Exemplary Engineered Cell

In some embodiments, an engineered cell comprises: (a) a first stably integrated heterologous polynucleic acid comprising the nucleic acid sequence encoding for Gag and the nucleic acid sequence encoding for Pol; (b) a second stably integrated heterologous polynucleic acid comprising the nucleic acid sequence encoding for Rev; and (c) a third stably integrated heterologous polynucleic acid comprising the nucleic acid sequence encoding for VSV-G.

In some embodiments, the first stably integrated heterologous polynucleic acid comprises a first expression cassette comprising: (i) a nucleic acid sequence of a first chemically inducible promoter (as described herein); and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). In some embodiments, the first chemically inducible promoter comprises the nucleic acid sequence of one or more of SEQ ID NOs: 1-9. In some embodiments, the first stably integrated heterologous polynucleic acid further comprises a nucleic acid sequence of a selection marker (as described herein) that is operably linked to a nucleic acid sequence of a promoter (constitutive or inducible, as described herein).

In some embodiments, the second stably integrated heterologous polynucleic acid comprises a second expression cassette comprising: (i) a nucleic acid sequence of a second chemically inducible promoter (as described herein); and (ii) the nucleic acid sequence encoding for Rev (or a functional variant thereof). In some embodiments, the second chemically inducible promoter comprises the nucleic acid sequence of one or more of SEQ ID NOs: 1-9. In some embodiments, the second stably integrated heterologous polynucleic acid further comprises a nucleic acid sequence of a selection marker (as described herein) that is operably linked to a nucleic acid sequence of a promoter (constitutive or inducible, as described herein). In some embodiments, the second stably integrated heterologous polynucleic acid comprises a nucleic acid sequence of a transcriptional activator operably linked to a nucleic acid sequence of a promoter, wherein the transcriptional activator, when expressed in the presence of a small molecule inducer, binds to a chemically inducible promoter of the lentiviral vector production system. In some embodiments, the transcriptional activator comprises the amino acid sequence of any one of SEQ ID NOs: 10-16.

In some embodiments, the third stably integrated heterologous polynucleic acid comprises a third expression cassette comprising: (i) a nucleic acid sequence of a third chemically inducible promoter (as described herein); and (ii) the nucleic acid sequence encoding for VSV-G (or a functional variant thereof). In some embodiments, the third chemically inducible promoter comprises the nucleic acid sequence of one or more of SEQ ID NOs: 1-9. In some embodiments, the third stably integrated heterologous polynucleic acid further comprises a nucleic acid sequence of a selection marker (as described herein) that is operably linked to a nucleic acid sequence of a promoter (constitutive or inducible, as described herein).

In some embodiments, the first chemically inducible promoter, the second chemically inducible promoter, and the third chemically inducible promoter comprise the same nucleic acid sequence.

In some embodiments, the first stably integrated polynucleic acid, the second stably integrated polynucleic acid, or the third stably integrated polynucleic acid comprises the additional polynucleic acid. In other embodiments, the additional polynucleic acid is a fourth stably integrated heterologous polynucleic acid of the engineered cell.

In some embodiments, each polynucleic acid of a lentiviral vector production system that is stably integrated into the genome of the engineered cell is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the engineered cell further comprises a stable landing pad (as described herein).

In some embodiments, the engineered cell is derived from a HEK293 cell, a HeLa cell, a BHK cell, or a Sf9 cell.

### 6. Sixth Exemplary Engineered Cell

In some embodiments, an engineered cell comprises: (a) a first stably integrated heterologous polynucleic acid comprising the nucleic acid sequence encoding for Gag and the nucleic acid sequence encoding for Pol; (b) a second stably integrated heterologous polynucleic acid comprising the nucleic acid sequence encoding for Rev; and (c) a third stably integrated heterologous polynucleic acid comprising the nucleic acid sequence encoding for VSV-G.

In some embodiments, the first stably integrated heterologous polynucleic acid comprises a first expression cassette comprising: (i) a nucleic acid sequence of a first exogenous promoter; and (ii) a nucleic acid sequence encoding for a polycistronic RNA, wherein the polycistronic RNA comprises the nucleic acid sequence encoding for Gag (or a functional variant thereof) and the nucleic acid sequence encoding for Pol (or a functional variant thereof). The skilled person will understand that the chemically inducible promoters described above and exogenous promoters may have the same sequence. For example, the tet-controlled transactivator (tTA) may bind to a promoter (e.g. an exogenous promoter or a chemically inducible promoter) in the absence of a small molecule inducer. Introduction of a small molecule repressor may decrease binding of tTA to the promoter.

In some embodiments, the first exogenous promoter comprises the nucleic acid sequence of one or more of SEQ ID NOs: 1-9. In some embodiments, the first stably integrated heterologous polynucleic acid further comprises a nucleic acid sequence of a selection marker (as described herein) that is operably linked to a nucleic acid sequence of a promoter (constitutive or inducible, as described herein).

In some embodiments, the second stably integrated heterologous polynucleic acid comprises a second expression cassette comprising: (i) a nucleic acid sequence of a second exogenous promoter (as described herein); and (ii) the nucleic acid sequence encoding for Rev (or a functional variant thereof). In some embodiments, the second exogenous promoter comprises the nucleic acid sequence of one or more of SEQ ID NOs: 1-9. In some embodiments, the second stably integrated heterologous polynucleic acid further comprises a nucleic acid sequence of a selection marker (as described herein) that is operably linked to a nucleic acid sequence of a promoter (constitutive or inducible, as described herein).

In some embodiments, the third stably integrated heterologous polynucleic acid comprises a third expression cassette comprising: (i) a nucleic acid sequence of a third exogenous promoter (as described herein); and (ii) the nucleic acid sequence encoding for VSV-G (or a functional variant thereof). In some embodiments, the third exogenous promoter comprises the nucleic acid sequence of one or more of SEQ ID NOs: 1-9. In some embodiments, the third stably integrated heterologous polynucleic acid further comprises a nucleic acid sequence of a selection marker (as described herein) that is operably linked to a nucleic acid sequence of a promoter (constitutive or inducible, as described herein).

In some embodiments, the first exogenous promoter, the second exogenous promoter, and the third chemically exogenous comprise the same nucleic acid sequence.

In some embodiments, the additional polynucleic acid is transiently transfected into the engineered cell. In some embodiments, the additional polynucleic acid is not stably integrated into the engineered cell. In some embodiments, the additional polynucleic acid is encoded on a plasmid or vector in the engineered cell. In some embodiments, the additional polynucleic acid comprises a sequencing encoding Tetracycline-controlled transactivator (tTA), which does not require small molecule binding to bind to an exogenous promoter, e.g., as described in Gossen et al. Proceedings of the National Academy of Sciences 89.12 (1992): 5547-5551, which is incorporated by reference in its entirety. In some embodiments, the additional polynucleic acid comprises a sequencing encoding Tetracycline-controlled transactivator (tTA), or a functional variant thereof. A functional variant of tTA comprises at least 80% identity (at least 85%, at least 90%, at least 95%, at least 96%, at least 97%, at least 98%, or at least 99% identity) with any one of SEQ ID NOs: 11 and 27-28 and maintains at least 80% of the function (at least 85%, at least 90%, at least 95%, at least 96%, at least 97%, at least 98%, or at least 99% of the function) of tTA (e.g., tTA-1 (that is, its function as a transcriptional activator). In some embodiments, the tTA is selected from the group consisting of SEQ ID NOs: 11 and 27-28.

In some embodiments, each polynucleic acid of a lentiviral vector production system that is stably integrated into the genome of the engineered cell is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the engineered cell further comprises a stable landing pad (as described herein).

In some embodiments, the engineered cell is derived from a HEK293 cell, a HeLa cell, a BHK cell, or a Sf9 cell.

### III. Kits

In some aspects, the disclosure relates to kits comprising a lentiviral vector production system described herein in Part I.

In some embodiments, a kit comprises one or more polynucleic acids collectively comprising a lentiviral vector production system.

In some embodiments, a kit comprises an engineered cell described in Part II.

In some embodiments, a kit comprises a transfer polynucleic acid. The transfer polynucleic acids described herein comprise a central nucleic acid sequence flanked, on the 5′ end and the 3′ end, by a nucleic acid sequence of a lentivirus long tandem repeat (LTR). Exemplary lentivirus LTRs are known to those having ordinary skill in the art.

The central nucleic acid of a transfer polynucleic acid may comprise a nucleic acid sequence of a multiple cloning site. Exemplary multiple cloning sites are known to those having ordinary skill in the art. A multiple cloning site can be used for cloning a payload molecule (or gene of interest)—or an expression cassette encoding a payload molecule—into the transfer polynucleic acid prior to the generation of viral vectors in a host cell.

In some embodiments, the kit comprises a nucleic acid sequence encoding a landing pad capable of being stably integrated into the engineered cell (as described in section II(A)). In some embodiments, the landing pad is comprised within a vector. In some embodiments, the transfer nucleic acid can be integrated into the landing pad.

In some embodiments, the kit comprises a nucleic acid sequence encoding an integrase that is operably linked to a promoter (constitutive or inducible as described herein). In some embodiments, the kit comprises a mRNA encoding an integrase. In some embodiments, the kit comprises integrase protein.

In some embodiments, a kit further comprises a small molecule inducer corresponding to a chemically inducible promoter of the lentiviral vector production. In some embodiments, a small molecule inducer is tetracycline, doxycycline, cumate, ABA, CRY2-CIB1, DAPG, mifepristone, lactose, or arabinose. In some embodiments, the kit comprising a polynucleic acid (e.g. a vector or plasmid) comprising a nucleic acid sequence encoding a Tetracycline-controlled transactivator (tTA) or variant thereof, as described herein.

In some embodiments, a kit comprises an engineered cell, wherein the engineered cell comprises a chemically inducible promoter comprising the nucleic acid sequence of one or more of SEQ ID NOs: 1-9 operably linked to the nucleic acid sequence encoding for Gag (or a functional variant thereof), the nucleic acid sequence encoding for Pol (or a functional variant thereof), the nucleic acid sequence encoding for VSV-G (or a functional variant thereof), the nucleic acid sequence encoding for Rev (or a functional variant thereof), or a combination thereof. In some embodiments, the nucleic acid sequence encoding for Gag (or a functional variant thereof), the nucleic acid sequence encoding for Pol (or a functional variant thereof), the nucleic acid sequence encoding for VSV-G (or a functional variant thereof), and the nucleic acid sequence encoding for Rev (or a functional variant thereof) are each operably linked to a chemically inducible promoter comprising the nucleic acid sequence of one or more of SEQ ID NOs: 1-9. In some embodiments, the engineered cell comprises at least two chemically inducible promoters. In some embodiments, two or more of the at least two chemically inducible promoters are distinct.

In some embodiments, a kit comprises an engineered cell, wherein the engineered cell comprises a chemically inducible promoter comprising the nucleic acid sequence of one or more of SEQ ID NOs: 1-3 operably linked to the nucleic acid sequence encoding for Gag (or a functional variant thereof), the nucleic acid sequence encoding for Pol (or a functional variant thereof), the nucleic acid sequence encoding for VSV-G (or a functional variant thereof), the nucleic acid sequence encoding for Rev (or a functional variant thereof), or a combination thereof. In some embodiments, the nucleic acid sequence encoding for Gag (or a functional variant thereof), the nucleic acid sequence encoding for Pol (or a functional variant thereof), the nucleic acid sequence encoding for VSV-G (or a functional variant thereof), and the nucleic acid sequence encoding for Rev (or a functional variant thereof) are each operably linked to a chemically inducible promoter comprising the nucleic acid sequence of one or more of SEQ ID NOs: 1-3. In some embodiments, the engineered cell comprises at least two chemically inducible promoters. In some embodiments, two or more of the at least two chemically inducible promoters are distinct.

In some embodiments, the kit comprises an engineered cell comprising a landing pad (as described in section II(A)). In some embodiments, the transfer nucleic acid can be integrated into the landing pad.

In some embodiments, a kit comprises a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the lentiviral vector production system, optionally wherein an engineered cell comprises the polynucleic acid comprising the nucleic acid sequence of the transcriptional activator. In some embodiments, the transcriptional activator comprises the amino acid sequence of any one of SEQ ID NOs: 10-16. In some embodiments, the kit comprises the small molecule inducer doxycycline, tetracycline, DAPG, cumate lactose, or arabinose.

In some embodiments, a kit comprises a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the lentiviral vector production system, optionally wherein an engineered cell comprises the polynucleic acid comprising the nucleic acid sequence of the transcriptional activator. In some embodiments, the transcriptional activator comprises the amino acid sequence of any one of SEQ ID NOs: 10-12. In some embodiments, the kit comprises the small molecule inducer doxycycline or tetracycline.

In some embodiments, a kit comprises a polynucleic acid comprising a nucleic acid sequence of a transcriptional activator operably linked to a nucleic acid sequence of a promoter, wherein the transcriptional activator binds to an exogenous promoter of the lentiviral vector production system, optionally wherein an engineered cell comprises the polynucleic acid comprising the nucleic acid sequence of the transcriptional activator (e.g., encoding a Tetracycline-controlled transactivator (tTA)).

### IV. Methods

In some aspects, the disclosure relates to methods of producing a lentiviral vector in an engineered cell (e.g., an engineered cell, as described herein). In some embodiments, the method comprises expressing Gag (or a functional variant thereof), Pol (or a functional variant thereof), VSV-G (or a functional variant thereof), and Rev (or a functional variant thereof) in the engineered cell.

In some embodiments, the engineered cell comprises an inducible lentiviral vector production system (as described herein). For example, in some embodiments, an engineered cell comprises a heterologous polynucleic acid comprising a nucleic acid sequence of a chemically inducible promoter (as described herein) that is operably linked to the nucleic acid sequence encoding for Gag (or a functional variant thereof), Pol (or a functional variant thereof), VSV-G (or a functional variant thereof), Rev (or a functional variant thereof), or a combination thereof. In some embodiments, the method comprises expressing a transcriptional activator that binds to the chemically inducible promoter and contacting the engineered cell with a small molecule inducer corresponding to the chemically inducible promoter. In some embodiments, the engineered cell is induced to uptake a heterologous polynucleic acid comprising the nucleic acid sequence of the transcriptional activator.

In some embodiments, the engineered cell comprises a chemically inducible promoter comprising the nucleic acid sequence of one or more of SEQ ID NOs: 1-3 operably linked to the nucleic acid sequence encoding for Gag (or a functional variant thereof), the nucleic acid sequence encoding for Pol (or a functional variant thereof), the nucleic acid sequence encoding for VSV-G (or a functional variant thereof), the nucleic acid sequence encoding for Rev (or a functional variant thereof), or a combination thereof.

In some embodiments, the nucleic acid sequence encoding for Gag (or a functional variant thereof), the nucleic acid sequence encoding for Pol (or a functional variant thereof), the nucleic acid sequence encoding for VSV-G (or a functional variant thereof), and the nucleic acid sequence encoding for Rev (or a functional variant thereof) are each operably linked to a chemically inducible promoter comprising the nucleic acid sequence of one or more of SEQ ID NOs: 1-9.

In some embodiments, the nucleic acid sequence encoding for Gag (or a functional variant thereof), the nucleic acid sequence encoding for Pol (or a functional variant thereof), the nucleic acid sequence encoding for VSV-G (or a functional variant thereof), and the nucleic acid sequence encoding for Rev (or a functional variant thereof) are each operably linked to a chemically inducible promoter comprising the nucleic acid sequence of one or more of SEQ ID NOs: 1-3.

In some embodiments, the engineered cell comprises at least two chemically inducible promoters. In some embodiments, two or more of the at least two chemically inducible promoters are distinct.

In some embodiments, the transcriptional activator comprises the amino acid sequence of any one of SEQ ID NOs: 10-16. In some embodiments, the small molecule inducer is doxycycline, tetracycline, DAPG, cumate, lactose, or arabinose.

In some embodiments, the transcriptional activator comprises the amino acid sequence of any one of SEQ ID NOs: 10-12. In some embodiments, the small molecule inducer is doxycycline or tetracycline.

In some embodiments, the engineered cell comprises an inducible lentiviral vector production system, wherein induction occurs when a polynucleic acid comprising a transcriptional activator (e.g., a transcriptional activator that is capable of binding an exogenous promoter in the absence of a small molecule repressor) is introduced into the engineered cells. For example, in some embodiments, an engineered cell comprises a heterologous polynucleic acid comprising a nucleic acid sequence of an exogenous promoter (as described herein) that is operably linked to the nucleic acid sequence encoding for Gag (or a functional variant thereof), Pol (or a functional variant thereof), VSV-G (or a functional variant thereof), Rev (or a functional variant thereof), or a combination thereof. In some embodiments, the method comprises expressing a transcriptional activator that binds to the exogenous promoter, e.g. the engineered cell is induced to uptake a heterologous polynucleic acid comprising the nucleic acid sequence of the transcriptional activator.

In some embodiments, the engineered cell comprises an exogenous promoter comprising the nucleic acid sequence of one or more of SEQ ID NOs: 1-3 operably linked to the nucleic acid sequence encoding for Gag (or a functional variant thereof), the nucleic acid sequence encoding for Pol (or a functional variant thereof), the nucleic acid sequence encoding for VSV-G (or a functional variant thereof), the nucleic acid sequence encoding for Rev (or a functional variant thereof), or a combination thereof.

In some embodiments, the nucleic acid sequence encoding for Gag (or a functional variant thereof), the nucleic acid sequence encoding for Pol (or a functional variant thereof), the nucleic acid sequence encoding for VSV-G (or a functional variant thereof), and the nucleic acid sequence encoding for Rev (or a functional variant thereof) are each operably linked to an exogenous promoter comprising the nucleic acid sequence of one or more of SEQ ID NOs: 1-9.

In some embodiments, the nucleic acid sequence encoding for Gag (or a functional variant thereof), the nucleic acid sequence encoding for Pol (or a functional variant thereof), the nucleic acid sequence encoding for VSV-G (or a functional variant thereof), and the nucleic acid sequence encoding for Rev (or a functional variant thereof) are each operably linked to an exogenous promoter comprising the nucleic acid sequence of one or more of SEQ ID NOs: 1-3.

In some embodiments, the engineered cell comprises at least two exogenous promoters. In some embodiments, two or more of the at least two exogenous promoters are distinct.

In some embodiments, the transcriptional activator is tet-controlled transactivator (tTA), which does not require a small molecule inducer to bind to an exogenous promoter, or a functional variant thereof (as described above).

In some embodiments, a polynucleic acid of a lentiviral vector production system is stably integrated into the genome at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies. In some embodiments, each polynucleic acid of a lentiviral vector production system that is stably integrated into the genome is stably integrated at a copy number of 10-25, 25-50, 25-100, 50-75, 50-100, 50-125, 50-150, 50-200, 75-100, 75-125, 75-150, 75-175, 75-200, 100-125, 100-150, 100-175, or 100-200 copies.

In some embodiments, the method further comprises introducing a transfer polynucleic acid into the engineered cell. In some embodiments, the transfer polynucleic acid is introduced before inducing the expression of Gag (or a functional variant thereof), Pol (or a functional variant thereof), VSV-G (or a functional variant thereof), and Rev (or a functional variant thereof). In some embodiments, the transfer polynucleic acid is introduced concurrently with inducing the expression of Gag (or a functional variant thereof), Pol (or a functional variant thereof), VSV-G (or a functional variant thereof), and Rev (or a functional variant thereof). In some embodiments, the transfer polynucleic acid is introduced after inducing the expression of Gag (or a functional variant thereof), Pol (or a functional variant thereof), VSV-G (or a functional variant thereof), and Rev (or a functional variant thereof).

### EXAMPLES

### Example 1. Inducible Control Over Lentivirus Vector Production

It was hypothesized that replacing the constitutive promoters (e.g., CMV) driving VSV-G, Gag-Pol, and Rev expression with an inducible promoter (e.g., a tetracycline responsive elements (TRE)) would allow for robust inducible expression of lentivirus in the presence of a small molecule inducer (e.g., doxycycline and a reverse tetracycline transactivator (rtTA) variant), while at the same time minimizing lentivirus production and associated toxicity in the absence of the small molecule inducer (FIGS. 1-3, 6-7). In an exemplary system, each gene resides on a separate plasmid, driven by TRE, and flanked by SB100X IR/DRs (FIGS. 2-3). The separation of viral genes into separate plasmids results in integration into separate parts of the genome, increasing safety by decreasing the ability to form competent lentiviral particles. SB100X IR/DRs allow for more efficient integration into the genome compared to random integration, which reduces the number of integration and selection steps required, since multiple integrations/selections can be performed simultaneously.

The TetOn variant uses mutations described in Das et al. Curr. Gene Ther. 2016; 16(3): 156-67. doi: 10.2174/1566523216666160524144041. TRE promoters were synthesized. Sleeping beauty IR/DRs and transposase were as described in Mates et al., Nat. Genet. 2009 June; 41(6): 753-61. doi: 10.1038/ng.343.

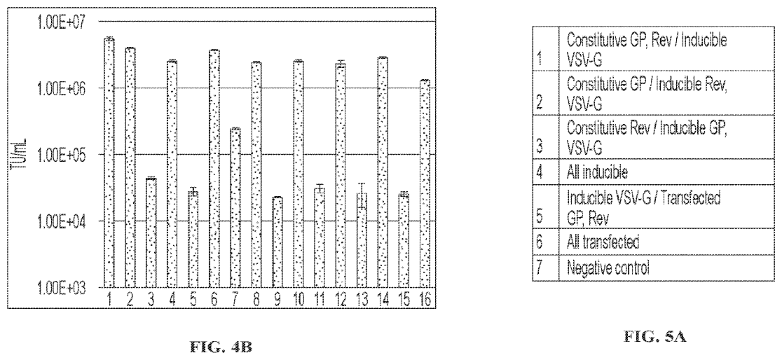

Plasmids were cotransfected into HEK293FT cells in combinations as listed in the transfection table (FIG. 4A), lentivirus was harvested at 48 hours and/or 72 hours after transfection, and lentivirus was titered by transduction onto HEK293FT cells and determining the percentage of EYFP-expressing cells 72 hours after transduction (FIG. 4B).

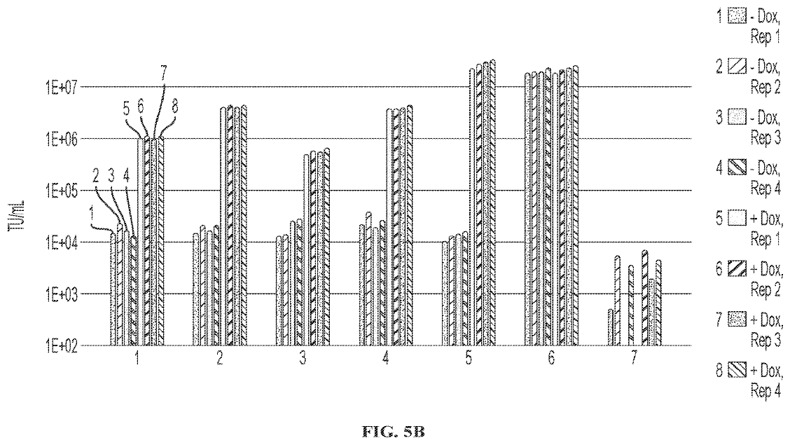

Several stable adherent HEK293T cell lines with lentiviral genes integrated into the genome were generated by cotransfection of plasmids encoding both lentiviral genes and antibiotic resistance genes, followed by selection using antibiotics. After selection and recovery, cells were transfected with only the LTR-containing lentiviral transfer plasmid and doxycycline was either provided or withheld (FIGS. 5A-5B).

Fully stable adherent HEK293T cells containing genomically integrated LTR-containing transfer sequence along with genomically integrated lentiviral genes were generated by cotransfection and antibiotic selection. These fully stable producer cells do not require addition of any further exogenous DNA prior to production of lentivirus, the addition of doxycycline produces all needed viral components for producing lentivirus. Following selection for about three weeks, cells were tested by plating at the same density, adding various concentrations of doxycycline, harvesting lentivirus 72 hours after induction, and transducing HEK293FT cells (FIG. 8). Transduction efficiency was measured using flow cytometry to determine the percentage of EYFP-expressing cells. Titers were compared to the traditional transfection production method using HEK293FT cells and negative control cells that were not transfected. In the absence of doxycycline, the stable producer cells produce minimal infectious titers, while in the presence of greater than 100 nM doxycycline, titers near those achievable with transient transfection were obtained. Titers from stable producer titers are approximately 86% those of the traditional transfection samples.

TABLE 1
Exemplary chemically inducible promoters/operators.

| SEQ | | |
|---|---|---|
| ID | | |
| NO: | Name | Sequence |
| 1 | pTREtight | CTCGAGTTTACTCCCTATCAGTGATAGAGAACGTATGTCGAGTTTACTCCCT |
| | | ATCAGTGATAGAGAACGATGTCGAGTTTACTCCCTATCAGTGATAGAGAAC |
| | | GTATGTCGAGTTTACTCCCTATCAGTGATAGAGAACGTATGTCGAGTTTACT |
| | | CCCTATCAGTGATAGAGAACGTATGTCGAGTTTATCCCTATCAGTGATAGAG |
| | | AACGTATGTCGAGTTTACTCCCTATCAGTGATAGAGAACGTATGTCGAGGTA |
| | | GGCGTGTACGGTGGGAGGCCTATATAAGCAGAGCTCGTTTAGTGAACCGTC |
| | | AGATCGCCTGGAGAATTCGAGCTCGGTACCCGGGGA |
| 2 | pTRE3G | GTTTACTCCCTATCAGTGATAGAGAACGTATGAAGAGTTTACTCCCTATCAG |
| | | TGATAGAGAACGTATGCAGACTTTACTCCCTATCAGTGATAGAGAACGTAT |
| | | AAGGAGTTTACTCCCTATCAGTGATAGAGAACGTATGACCAGTTTACTCCCT |
| | | ATCAGTGATAGAGAACGTATCTACAGTTTACTCCCTATCAGTGATAGAGAAC |
| | | GTATATCCAGTTTACTCCCTATCAGTGATAGAGAACGTATAAGCTTTAGGCG |
| | | TGTACGGTGGGCGCCTATAAAAGCAGAGCTCGTTTAGTGAACCGTCAGATCG |
| | | CCTGGAGCAATTCCACAACACTTTTGTCTTATACCAACTTTCCGTACCACTT |
| | | CCTACCCTCGTAAAGTCGACACCGGGGCCCAGATCTATCGATCGGCCGGAT |
| | | AACGCCACC |
| 3 | bi-TRE3G | GAATTCTCCAGGCGATCTGACGGTTCACTAAACGAGCTCTGCTTATATAGGC |
| | | CTCCCACCGTACACGCCACCTCGACATACTCGAGTTTACTCCCTATCAGTGA |
| | | TAGAGAACGTATGAAGAGTTTACTCCCTATCAGTGATAGAGAACGTATGCA |
| | | GACTTTACTCCCTATCAGTGATAGAGAACGTATAAGGAGTTTACTCCCTATC |
| | | AGTGATAGAGAACGTATGACCAGTTTACTCCCTATCAGTGATAGAGAACGT |
| | | ATCTACAGTTTACTCCCTATCAGTGATAGAGAACGTATATCCAGTTTACTCC |
| | | CTATCAGTGATAGAGAACGTATAAGCTTTAGGCGTGTACGGTGGGCGCCTA |
| | | TAAAAGCAGAGCTCGTTTAGTGAACCGTCAGATCGCCTGGAGCAATTCCACA |
| | | ACACTTTTGTCTTATACCAACTTTCCGTACCACTTCCTACCCTCGTAAAGTC |
| | | GACACCGGGGCCCAGATCTCCGCGGGGATCC |
| 4 | VanR | ATTGGATCCAAT |
| | operator | |
| 5 | TtgR | TATTTACAAACAACCATGAATGTAAGTA |
| | operator | |
| 6 | Gal4 UAS | CGGAGTACTGTCCTCCGA |
| | (for CID | |
| | systems) | |
| 7 | PhlF | ATGATACGAAACGTACCGTATCGTTAAGGT |
| | operator | |
| 8 | CymR | AGAAACAAACCAACCTGTCTGTATTA |
| | operator | |
| | v1 | |
| 9 | CymR | AACAAACAGACAATCTGGTCTGTTTGTA |
| | operator | |
| | v2 | |

TABLE 2
Exemplary transcriptional activators.

| SEQ | | |
|---|---|---|
| ID | | |
| NO: | Name | Sequence |
| 10 | Tet-On | MSRLDKSKVINSALELLNGVGIEGLTTRKLAQKLGVEQPTLYWHVKNKRALLD |
| | 3G | ALPIEMLDRHHTHSCPLEGESWQDFLRNNAKSYRCALLSHRDGAKVHLGTRPT |
| | | EKQYELTLENQLAFLCQQGFSLENALYALSAVGHFTLGCVLEEQEHQVAKEER |
| | | ETPTTDSMPPLLKQAIELFDRQGAEPAFLFGLELIICGLEKQLKCESGGPTDA |
| | | LDDFDLDMLPADALDDFDLDMLPADALDDFDLDMLPG |
| 11 | TetOff- | MSRLDKSKVINSALELLNEVGIEGLTTRKLAQKLGVEQPTLYWHVKNKRALLD |
| | Advanced | ALAIEMLDRHHTHFCPLEGESWQDFLRNNAKSFRCALLSHRDGAKVHLGTRPT |
| | (tTA-v1) | EKQYETLENQLAFLCQQGFSLENALYALSAVGHFTLGCVLEDQEHQVAKEERE |
| | | TPTTDSMPPLLRQAIELFDHQGAEPAFLFGLELIICGLEKQLKCESGGPADAL |
| | | DDFDLDMLPADALDDFDLDMLPADALDDFDLDMLPG |
| 12 | VanR- | MDMPRIKPGQRVMMALRKMIASGEIKSGERIAEIPTAAALGVSRMPVRIALRSL |
| | VP16 | EQEGLVVRLGARGYAARGVSSDQIRDAIEVRGVLEGFAARRLAERGMTAETHA |
| | | RFVVLIAEGEALFAAGRLNGEDLDRYAAYNQAFHDTLVSAAGNGAVESALAR |
| | | NGFEPFAAAGALALDLMDLSAEYEHLLAAHRQHQAVLDAVSCGDAEGAERIM |
| | | RDHALAAIRNAKVFEAAASAGAPLGAAWSIRADSGGGGPTDALDDFDLDMLP |
| | | ADALDDFDLDMLPADALDDFDLDMLPGPPKKKRKV |
| 13 | TtgR- | MVRRTKEEAQETRAQIIEAAERAFYKRGVARTTLADIAELAGVTRGAIYWHEN |
| | VP16 | NKAELVQALLDSLHETHDHLARASESEDEVDPLGCMRKLLLQVFNELVLDART |
| | | RRINEILHHKCEFTDDMCEIRQQHQSAVLDCHKGITLTLANVVRRGQLPGELDA |
| | | ERAAVAMFAYVDGLIRRWLLLPDSVDLLGDVEKWVDTGLDMLRLSPALRKSG |
| | | GGGPTDALDDFDLDMLPADALDDFDLDMLPADALDDFDLDMLPGPPKKKRKV |
| 14 | PhlF- | MARTPSRSSIGSLRSPHTHKAILTSTIEILKECGYSGLSIESVARRAGAGKPTIY |
| | VP16 | RWWTNKAALIAEVYENEIEQVRKFPDLGSFKADLDFLLHNLWKVWRETICGEAF |
| | | RCVIAEAQLDPVTLTQLKDQFMERRREIPKKLVEDAISNGELPKDINRELLLDMI |
| | | FGFCWYRLLTEQLTVEQDIEEFTFLLINGVCPGTQCSGGGGPTDALDDFDLDML |
| | | PADALDDFDLDMLPADALDDFDLDMLPGPPKKKRKV |
| 15 | CTA | MSPKRRTQAERAMETQGKLIAAALGVLREKGYAGFRIADVPGAAGVSRGAQS |
| | | HHFPTKLELLLATFEWLYEQITERSRARLAKLKPEDDVIQQMLDDAAEFFLDDD |
| | | FSIGLDLIVAADRDPALREGIQRTVERNRFVVEDMWLGVLVSRGLSRDDAEDIL |
| | | WLIFNSVRGLVVRSLWQKDKERFERVRNSTLEIARERYAKFKRSGGGGPTDAL |
| | | DDFDLDMLPADALDDFDLDMLPADALDDFDLDMLPGPPKKKRKV |
| 16 | rcTA | MVIMSPKRRTQAERAMETQGKLIAAALGVLREKGYAGFRIADVPGAAGVSRG |
| | (reverse | AQSHHFPTKLELLLATFEWLYEQITERSRARLAKLKPEDDVIQQMLDDAAEFFL |
| | CymR | DDDFSIGLDLIVAADRDPVLREGIQRTVERNRFVVGDIWLGVLVSRGLSRDDAE |
| | fused to | DILWLIFNSVRGLVVRSLWQKDKERFERVRNSTLEIARERYAKFKRSGGGGPTD |
| | 3x VP16 | ALDDFDLDMLPADALDDFDLDMLPADALDDFDLDMLPGPPKKKRKV** |
| | and an | |
| | NLS) | |
| 27 | tTA-v2 | MSRLDKSKVINSALELLNEVGIEGLTTRKLAQKLGVEQPTLYWHVKNKRALLD |
| | | ALAIEMLDRHHTHFCPLEGESWQDFLRNNAKSFRCALLSHRDGAKVHLGTRPT |
| | | EKQYETLENQLAFLCQQGFSLENALYALSAVGHFTLGCVLEDQEHQVAKEERE |
| | | TPTTDSMPPLLRQAIELFDHQGAEPAFLFGLELIICGLEKQLKCESGSPKKKR |
| | | KVDYKDDDDKPADALDDFDLDMLPADALDDFDLDMLPADALDDFDLDML |
| 28 | tTA-v3 | MSRLDKSKVINSALELLNEVGIEGLTTRKLAQKLGVEQPTLYWHVKNKRALLD |
| | | ALAIEMLDRHHTHFCPLEGESWQDFLRNNAKSFRCALLSHRDGAKVHLGTRPT |
| | | EKQYETLENQLAFLCQQGFSLENALYALSAVGHFTLGCVLEDQEHQVAKEERE |
| | | TPTTDSMPPLLRQAIELFDHQGAEPAFLFGLELIICGLEKQLKCESGSAYSRA |
| | | RTKNNYGSTIEGLLDLPDDDAPEEAGLAAPRLSFLPAGHTRRLSTAPPTDVSL |
| | | GDELHLDGEDVAMAHADALDDFDLDMLGDGDSPGPGFTPHDSAPYGALDMADF |
| | | EFEQMFTDALGIDEYGG |

TABLE 3
Exemplary lentiviral vector production components.

| SEQ | | |
|---|---|---|
| ID | | |
| NO: | Name | Sequence |
| 17 | Gag | MGARASVLSGGELDRWEKIRLRPGGKKKYKLKHIVWASRELERFAVNPGLLET |
| | | SEGCRQILGQLQPSLQTGSEELRSLYNTVATLYCVHQRIEIKDTKEALDKIEE |
| | | EQNKSKKKAQQAAADTGHSNQVSQNYPIVQNIQGQMVHQAISPRTLNAWVKVV |
| | | EEKAFSPEVIPMFSALSEGATPQDLNTMLNTVGGHQAAMQMLKETINEEAAEW |
| | | DRVHPVHAGPIAPGQMREPRGSDIAGTTSTLQEQIGWMTHNPPIPVGEIYKRW |
| | | IILGLNKIVRMYSPTSILDIRQGPKEPFRDYVDRFYKTLRAEQASQEVKNWMT |
| | | ETLLVQNANPDCKTILKALGPGATLEEMMTACQGVGGPGHKARVLAEAMSQVT |
| | | NPATIMIQKGNFRNQRKTVKCFNCGKEGHIAKNCRAPRKKGCWKCGKEGHQMK |
| | | DCTERQAN |
| 18 | Pol | FFREDLAFPQGKAREFSSEQTRANSPTRRELQVWGRDNNSLSEAGADRQGTVSF |
| | | SFPQITLWQRPLVTIKIGGQLKEALLDTGADDTVLEEMNLPGRWKPKMIGGIGG |
| | | FIKVRQYDQILIEICGHKAIGTVLVGPTPVNIIGRNLLTQIGCTLNFPISPIET |
| | | VPVKLKPGMDGPKVKQWPLTEEKIKALVEICTEMEKEGKISKIGPENPYNTPVF |
| | | AIKKKDSTKWRKLVDFRELNKRTQDFWEVQLGIPHPAGLKQKKSVTVLDVGDAY |
| | | FSVPLDKDFRKYTAFTIPSINNETPGIRYQYNVLPQGWKGSPAIFQCSMTKILE |
| | | PFRKQNPDIVIYQYMDDLYVGSDLEIGQHRTKIEELRQHLLRWGFTTPDKKHQK |
| | | EPPFLWMGYELHPDKWTVQPIVLPEKDSWTVNDIQKLVGKLNWASQIYAGIKVR |
| | | QLCKLLRGTKALTEVVPLTEEAELELAENREILKEPVHGVYYDPSKDLIAEIQK |
| | | QGQGQWTYQIYQEPFKNLKTGKYARMKGAHTNDVKQLTEAVQKIATESIVIWGK |
| | | TPKFKLPIQKETWEAWWTEYWQATWIPEWEFVNTPPLVKLWYQLEKEPIIGAE |
| | | TFYVDGAANRETKLGKAGYVTDRGRQKVVPLTDTTNQKTELQAIHLALQDSGL |
| | | EVNIVTDSQYALGIIQAQPDKSESELVSQIIEQLIKKEKVYLAWVPAHKGIGGN |
| | | EQVDKLVSAGIRKVLFLDGIDKAQEEHEKYHSNWRAMASDFNLPPVVAKEIVA |
| | | SCDKCQLKGEAMHGQVDCSPGIWQLDCTHLEGKVILVAVHVASGYIEAEVIPA |
| | | ETGQETAYFLLKLAGRWPVKTVHTDNGSNFTSTTVKAACWWAGIKQEFGIPYN |
| | | PQSQGVIESMNKELKKIIGQVRDQAEHLKTAVQMAVFIHNFKRKGGIGGYSAG |
| | | ERIVDIIATDIQTKELQKQITKIQNFRVYYRDSRDPVWKGPAKLLWKGEGAVVI |
| | | QDNSDIKVVPRRKAKIIRDYGKQMAGDDCVASRQDED |
| 19 | VSV-G | MKCLLYLAFLFIGVNCKFTIVFPHNQKGNWKNVPSNYHYCPSSSDLNWHNDLI |
| | | GTALQVKMPKSHKAIQADGWMCHASKWVTTCDFRWYGPKYITHSIRSFTPSVE |
| | | QCKESIEQTKQGTWLNPGFPPQSCGYATVIDAEAVIVQVTPHHVLVDEYTGEWV |
| | | DSQFINGKCSNYICPTVHNSTTWHSDYKVKGLCDSNLISMDITFFSEDGELSSL |
| | | GKEGTGFRSNYFAYETGGKACKMQYCKHWGVRLPSGVWFEMADKDLFAAARFPE |
| | | CPEGSSISAPSQTSVDVSLIQDVERILDYSLCQETWSKIRAGLPISPVDLSYLA |
| | | PKNPGTGPAFTIINGTLKYFETRYIRVDIAAPILSRMVGMISGTTTERELWDDWA |
| | | PYEDVEIGPNGVLRTSSGYKFPLYMIGHGMLDSDLHLSSKAQVFEHPHIQDAAS |
| | | QLPDDESLFFGDTGLSKNPIELVEGWFSSWKSSIASFFFIIGLIIGLFLVLRVGI |
| | | HLCIKLKHTKKRQIYTDIEMNRLGK |
| 20 | Rev | MAGRSGDSDEDLLKAVRLIKFLYQSNPPPNPEGTRQARRNRRRRWRERQRQIHS |
| | | ISERILSTYLGRSAEPVPLQLPPLERLTLDCNEDCGTSGTQGVGSPQILVESPT |
| | | ILESGAKE |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

1. An engineered cell for lentiviral vector production comprising one or more stably integrated heterologous polynucleic acids collectively comprising: a nucleic acid sequence encoding for Gag; a nucleic acid sequence encoding for Pol; a nucleic acid sequence encoding for VSV-G; and a nucleic acid encoding for Rev; at least one of which is operably linked to a chemically inducible promoter.

2. The engineered cell of claim 1, wherein the chemically inducible promoter comprises the nucleic acid sequence of one or more of SEQ ID NOs: 1-9.

3. The engineered cell of claim 1 or claim 2, wherein the engineered cell comprises a nucleic acid sequence encoding for Gag operably linked to a chemically inducible promoter.

4. The engineered cell of claim 3, wherein Gag comprises the amino acid sequence of SEQ ID NO: 17.

5. The engineered cell of any one of claims 1-4, wherein the engineered cell comprises a nucleic acid sequence encoding for Pol operably linked to a chemically inducible promoter.

6. The engineered cell of claim 5, wherein Pol comprises the amino acid sequence of SEQ ID NO: 18.

7. The engineered cell of any one of claims 1-6, wherein the engineered cell comprises a nucleic acid sequence encoding for VSV-G operably linked to a chemically inducible promoter.

8. The engineered cell of claim 7, wherein VSV-G comprises the amino acid sequence of SEQ ID NO: 19.

9. The engineered cell of any one of claims 1-8, wherein the engineered cell comprises a nucleic acid sequence encoding for Rev operably linked to a chemically inducible promoter.

10. The engineered cell of claim 9, wherein Rev comprises the amino acid sequence of SEQ ID NO: 20.

11. The engineered cell of any one of claims 1-10, wherein the engineered cell comprises:
(a) a first stably integrated heterologous polynucleic acid comprising the nucleic acid sequence encoding for Gag and the nucleic acid sequence encoding for Pol;
(b) a second stably integrated heterologous polynucleic acid comprising the nucleic acid sequence encoding for VSV-G; and
(c) a third stably integrated heterologous polynucleic acid comprising the nucleic acid sequence encoding for Rev.

12. The engineered cell of claim 11, wherein the first stably integrated heterologous polynucleic acid comprises a first expression cassette comprising: (i) a nucleic acid sequence of a first chemically inducible promoter; and (ii) a nucleic acid sequence encoding for a polycistronic RNA, wherein the polycistronic RNA comprises the nucleic acid sequence encoding for Gag and the nucleic acid sequence encoding for Pol.

13. The engineered cell of claim 12, wherein the first chemically inducible promoter comprises the nucleic acid sequence of one or more of SEQ ID NOs: 1-9.

14. The engineered cell of any one of claims 11-13, wherein the first stably integrated heterologous polynucleic acid further comprises a nucleic acid sequence of a selection marker that is operably linked to a nucleic acid sequence of a promoter.

15. The engineered cell of any one of claims 11-14, wherein the second stably integrated heterologous polynucleic acid comprises a second expression cassette comprising: (i) a nucleic acid sequence of a second chemically inducible promoter; and (ii) the nucleic acid sequence encoding for VSV-G.

16. The engineered cell of claim 15, wherein the second stably integrated heterologous polynucleic acid further comprises a nucleic acid sequence of a transcriptional activator operably linked to a nucleic acid sequence of a promoter, wherein the transcriptional activator, when expressed in the presence of a small molecule inducer, binds to the second chemically inducible promoter of the second expression cassette.

17. The engineered cell of claim 15 or claim 16, wherein the second chemically inducible promoter comprises the nucleic acid sequence of one or more of SEQ ID NOs: 1-9.

18. The engineered cell of any one of claims 11-17, wherein the second stably integrated heterologous polynucleic acid further comprises a nucleic acid sequence of a selection marker that is operably linked to a nucleic acid sequence of a promoter.

19. The engineered cell of any one of claims 11-18, wherein the third stably integrated heterologous polynucleic acid comprises a third expression cassette comprising: (i) a nucleic acid sequence of a third chemically inducible promoter; and (ii) the nucleic acid sequence encoding for Rev.

20. The engineered cell of claim 19, wherein the third stably integrated heterologous polynucleic acid further comprises a nucleic acid sequence of a transcriptional activator operably linked to a nucleic acid sequence of a promoter, wherein the transcriptional activator, when expressed in the presence of a small molecule inducer, binds to the third chemically inducible promoter of the third expression cassette.

21. The engineered cell of claim 19 or claim 20, wherein the third chemically inducible promoter comprises the nucleic acid sequence of one or more of SEQ ID NOs: 1-9.

22. The engineered cell of any one of claims 11-21, wherein the third stably integrated heterologous polynucleic acid further comprises a nucleic acid sequence of a selection marker that is operably linked to a nucleic acid sequence of a promoter.

23. The engineered cell of any one of claims 11-22, further comprising a heterologous polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the engineered cell.

24. The engineered cell of claim 23, wherein the transcriptional activator comprises the amino acid sequence of any one of SEQ ID NOs: 10-16.

25. The engineered cell of any one of claims 1-10, wherein the engineered cell comprises:
(a) a first stably integrated heterologous polynucleic acid comprising the nucleic acid sequence encoding for Gag and the nucleic acid sequence encoding for Pol; and
(b) a second stably integrated heterologous polynucleic acid comprising the nucleic acid sequence encoding for VSV-G and the nucleic acid sequence encoding for Rev.

26. The engineered cell of claim 25, wherein the first stably integrated heterologous polynucleic acid comprises a first expression cassette comprising: (i) a nucleic acid sequence of a first chemically inducible promoter; and (ii) a nucleic acid sequence encoding for a polycistronic RNA, wherein the polycistronic RNA comprises the nucleic acid sequence encoding for Gag and the nucleic acid sequence encoding for Pol.

27. The engineered cell of claim 26, wherein the first chemically inducible promoter comprises the nucleic acid sequence of one or more of SEQ ID NOs: 1-9.

28. The engineered cell of any one of claims 22-27, wherein the first stably integrated heterologous polynucleic acid further comprises a nucleic acid sequence of a selection marker that is operably linked to a nucleic acid sequence of a promoter.

29. The engineered cell of any one of claims 22-28, wherein the second stably integrated heterologous polynucleic acid comprises:
a second expression cassette comprising: (i) a nucleic acid sequence of a second chemically inducible promoter; and (ii) the nucleic acid sequence encoding for VSV-G; and
a third expression cassette comprising: (i) a nucleic acid sequence of a third chemically inducible promoter; and (ii) the nucleic acid sequence encoding for Rev.

30. The engineered cell of claim 29, wherein: the second promoter comprises the nucleic acid sequence of one or more of SEQ ID NOs: 1-9; the third promoter comprises the nucleic acid sequence of one or more of SEQ ID NOs: 1-9; or a combination thereof.

31. The engineered cell of any one of claims 24-30, wherein the second stably integrated heterologous polynucleic acid further comprises a nucleic acid sequence of a selection marker that is operably linked to a nucleic acid sequence of a promoter.

32. The engineered cell of any one of claims 24-30, further comprising a heterologous polynucleic acid comprising a nucleic acid sequence of a transcriptional activator operably linked to a nucleic acid sequence of a promoter, wherein the transcriptional activator, when expressed in the presence of a small molecule inducer, binds to a chemically inducible promoter of the engineered cell.

33. The engineered cell of claim 32, wherein the transcriptional activator comprises the amino acid sequence of any one of SEQ ID NOs: 10-16.

34. The engineered cell of any one of claims 1-33, further comprising a stable landing pad.

35. The engineered cell of any one of claims 1-34, wherein the engineered cell is derived from a HEK293 cell, a HeLa cell, a BHK cell, or a Sf9 cell.

36. A kit comprising the engineered cell of any one of claims 1-35.

37. The kit of claim 36, further comprising a transfer polynucleic acid molecule comprising, from 5′ to 3′: (i) a nucleic acid sequence of a 5′ lentivirus long tandem repeat (LTR); (ii) a multiple cloning site; and (iii) a nucleic acid sequence of a 3′ lentivirus long tandem repeat (LTR).

38. The kit of claim 37, wherein the transfer polynucleic acid is a plasmid or a vector.

39. The kit of any one of claims 36-38, wherein the kit further comprises a small molecule inducer corresponding to a chemically inducible promoter of the engineered cell.

40. The kit of any one of claims 36-39, wherein the engineered cell comprises a chemically inducible promoter comprising the nucleic acid sequence of one or more of SEQ ID NOs: 1-9 operably linked to the nucleic acid sequence encoding for Gag, the nucleic acid sequence encoding for Pol, the nucleic acid sequence encoding for VSV-G, the nucleic acid sequence encoding for Rev, or a combination thereof.

41. The kit of any one of claims 36-40, wherein the nucleic acid sequence encoding for Gag, the nucleic acid sequence encoding for Pol, the nucleic acid sequence encoding for VSV-G, and the nucleic acid sequence encoding for Rev are each operably linked to a chemically inducible promoter comprising the nucleic acid sequence of one or more of SEQ ID NOs: 1-9.

42. The kit of claim 41, wherein the engineered cell comprises at least two chemically inducible promoters.

43. The kit of claim 42, wherein two or more of the at least two chemically inducible promoters are distinct.

44. The kit of any one of claims 36-43, wherein the kit comprise a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell, optionally wherein the engineered cell comprises the polynucleic acid comprising the nucleic acid sequence of the transcriptional activator.

45. The kit of claim 44, wherein the transcriptional activator comprises the amino acid sequence of any one of SEQ ID NOs: 10-12.

46. The kit of claim 44 or claim 45, wherein the kit comprises the small molecule inducer doxycycline or tetracycline.

47. A method of producing a lentiviral vector comprising:
(a) introducing a transfer polynucleic acid into the engineered cell of any one of claims 1-35; and
(b) contacting the engineered cell with a small molecule inducer corresponding to a chemically inducible promoter of the engineered cell, thereby inducing expression of Gag, Pol, VSV-G, and Rev;
wherein the engineered cell comprises a heterologous polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the engineered cell;
wherein (b) occurs before, concurrently with, or after (a).

48. The method of claim 47, wherein the engineered cell comprises a chemically inducible promoter comprising the nucleic acid sequence of one or more of SEQ ID NOs: 1-3 operably linked to the nucleic acid sequence encoding for Gag, the nucleic acid sequence encoding for Pol, the nucleic acid sequence encoding for VSV-G, the nucleic acid sequence encoding for Rev, or a combination thereof.

49. The method of claim 47 or claim 48, wherein the nucleic acid sequence encoding for Gag, the nucleic acid sequence encoding for Pol, the nucleic acid sequence encoding for VSV-G, and the nucleic acid sequence encoding for Rev are each operably linked to a chemically inducible promoter comprising the nucleic acid sequence of one or more of SEQ ID NOs: 1-3.

50. The method of claim 49, wherein the engineered cell comprises at least two chemically inducible promoters.

51. The method of claim 50, wherein two or more of the at least two chemically inducible promoters are distinct.

52. The method of any one of claims 47-51, wherein the transcriptional activator comprises the amino acid sequence of any one of SEQ ID NOs: 10-12.

53. The method of any one of claims 47-52, wherein the small molecule inducer is doxycycline or tetracycline.

54. A method of producing a lentiviral vector in an engineered cell, wherein the engineered cell comprises one or more stably integrated heterologous polynucleic acids collectively comprising: a nucleic acid sequence encoding for Gag; a nucleic acid sequence encoding for Pol; a nucleic acid sequence encoding for VSV-G; and a nucleic acid encoding for Rev; at least one of which is operably linked to an exogenous promoter that is capable of being bound by an exogenous transcriptional activator in the absence of a small molecule repressor, said method comprising:
(a) introducing a transfer polynucleic acid into the engineered cell; and
(b) introducing the exogenous transcriptional activator into the engineered cell; and
(c) culturing the cell in the absence of the small molecule repressor, thereby inducing expression of Gag, Pol, VSV-G, and Rev;
wherein (b) occurs before, concurrently with, or after (a).

55. The method of claim 54, wherein the introducing in (b) is performed in the presence of the small molecule repressor.

56. The method of claim 54 or claim 55, wherein the introducing in (b) is performed by:
introducing a heterologous polynucleic acid into the cell, wherein the heterologous polynucleic acid comprises a nucleic acid sequence of the exogenous transcriptional activator operably linked to a nucleic acid of a promoter; and expressing the exogenous transcriptional activator.

57. The method of any one of claims 54-56, wherein the exogenous promoter comprises the nucleic acid sequence of one or more of SEQ ID NOs: 1-3.

58. The method of any one of claims 54-57, wherein the nucleic acid sequence encoding for Gag, the nucleic acid sequence encoding for Pol, the nucleic acid sequence encoding for VSV-G, and the nucleic acid sequence encoding for Rev are each operably linked to an exogenous promoter comprising the nucleic acid sequence of one or more of SEQ ID NOs: 1-3.

59. The method of claim 58, wherein the engineered cell comprises at least two exogenous promoters.

60. The method of claim 59, wherein two or more of the at least two exogenous promoters are distinct.

61. The method of any one of claims 54-60, wherein the transcriptional activator is Tetracycline-controlled transactivator (tTA).

62. The method of claim 61, wherein the tTA comprises the amino acid sequence of one or more of SEQ ID NOs: 11 and 27-28.
